(19) United States
(12) Patent Application Publication
(10) Pub. No.: US 2005/0272029 A1
(43) Pub. Date: Dec. 8, 2005

(54) Hepatitis c viral-like particle purification

(76) Inventors: Bertrand Saunier, Bethesda, MD (US); Miriam Triyatni, Bethesda, MD (US)

Correspondence Address: BERENATO, WHITE & STAVISH, LLC, 6550 ROCK SPRING DRIVE, SUITE 240, BETHESDA, MD, 20817, US

(21) Appl. No.: 10/524,443

(22) PCT Filed: Aug. 18, 2003
(86) PCT No.: PCT/US03/25674

§ 371 Date: May 18, 2005

Related U.S. Application Data
(60) Provisional application No. 60/404,183, filed on Aug. 16, 2002.

(51) Int. Cl. C12Q 1/70; C12Q 1/68

(52) U.S. Cl. 435/5; 435/6; 435/239

(57) ABSTRACT

Methods for obtaining HCV complexes and HCV-like particles comprising HCV structural genes are provided. In one method, cells containing HCV-like particles are lysed with digitonin in the presence of protease inhibitors. Polyethylene glycol is slowly added to the lysate, to provide a precipitate that comprises complexes of the HCV structural proteins associated with lipid vesicles or micelles and complexes comprising viral structural proteins in the form of insoluble aggregates. In another method, the lysate is centrifuged through a sucrose cushion. Preferably, the pellet is then subjected to equilibrium ultracentrifugation, to provide a preparation of HCV-like particles that are heterogenous in size. The third method comprises subjecting the infected cells to hypertonic/hypotonic shock, and lysing the cells with digitonin in the presence of protease inhibitors. The lysate is pelleted and fractionated to provide a population of HCV-like particles that are substantially homogenous and have an average diameter of about 50 nm. As used herein the term “substantially homogenous” means that the shape of the particles are similar and that the size of the particles vary by 10% or less. Methods of using the HCV complexes and HCV-Iike particles as screening tools, diagnostic tools, and immunogenic compositions are also provided. Methods of treating patients exhibiting symptoms of HCV infection with compounds or substances that interfere with binding or internalization of the present HCV-like particles to asialoglycoprotein receptors are also provided.

This application claims the benefit of U.S. Provisional Patent Application No. 404,183 filed Aug. 16, 2002, which is incorporated herein by reference.

This invention is supported, at least in part, by funding from the National Institutes of Health, USA. The U.S. government has certain rights in the invention.

### FIELD OF THE INVENTION

The invention relates to hepatitis C virus-like particles, a method for purifying the particles, methods of screening for the presence of hepatitis C virus, methods for screening compounds that interfere with binding and/or internalization of the virus-like particles to/into host cells, cell lines used for screening of the compounds, methods for detecting and identifying cellular receptors for hepatitis C virus and use of the hepatitis C virus-like particles to induce an immune reaction in an animal.

### BACKGROUND

Hepatitis C Virology

Hepatitis C virus (HCV) is an enveloped, positive-strand RNA virus belonging to the genus Hepacivirus and family Flaviviridae. HCV is classified into six major genotypes and ˜100 subtypes. The viral genome (˜9.6 kb) is translated into a single polyprotein of ˜3,000 amino acids. A combination of host and viral proteases are involved in polyprotein processing to give at least nine different proteins. This precursor is processed during and after translation to yield the mature structural (core, E1 and E2-p7) and non-structural NS2, NS3, NS4A, NS4B, NS5A and NS5B) proteins. The structural proteins of HCV are believed to comprise the core protein (˜21 kDa), and two envelope glycoproteins, E1 (˜31 kDa) and E2 (˜70 kDa).

E1 and E2 proteins are thought to play a role in the HCV life cycle, both in the assembly of infectious particles and in the initiation of viral infection by binding to its cellular receptor(s). Expression of recombinant E1 and E2 proteins in mammalian cells has shown that they associate into heterodimers. Both proteins are glycosylated and lack sialic acid at the termini of their carbohydrate domain in mammalian cells and probably in insect cells. Yet, it is not known whether these proteins form heterodimers at the surface of viral particles. In other enveloped viruses, a major role of envelope proteins is to bind to cellular receptor(s) and facilitate virus entry, thereby contributing in determining viral tropism.

E2 protein has also been implicated in the viral evasion from the immune system. Sequence analyses of different HCV isolates and sequential studies of virus isolates from infected patients suggest that the highly variable region 1 (HVR-1) in the amino-terminus of E2 protein is under immune selective pressure resulting in the selection of variants within the HVR-1. Previous studies have shown that antibodies specific for HVR-1 are neutralizing. However, these antibodies tend to be isolate-specific and over time drive the selection of new viral variants that are not recognized by the preexisting antibodies. Likewise, E2 protein may contribute to HCV resistance to interferon and impair natural killer (NK) cell function. The carboxy-terminal part of E2, p7, is generally cleaved, but only partially in some strains of genotype 1a. Although recent studies suggested that p7 might assist virion assembly and secretion from infected cells, its function remains unknown.

Studies have shown that HCV particles vary in size, between 30 to 60 nm in diameter. In addition, HCV particles display significant heterogeneity in buoyant density on sucrose density-gradient centrifugation, ranging from low (<1.07 g/ml) to high (1.25 g/ml) density. The heterogeneity of particle density has been attributed to the variability in size, non-enveloped nucleocapsid particles, and association with antibodies or β-lipoproteins.

Disease

HCV is the major etiology of non-A, non-B hepatitis that infects an estimated 170 millions people worldwide. One of its major characteristics is the high incidence of persistent infection, which may lead to autoimmune disorders and severe liver damage ranging from chronic hepatitis to liver cirrhosis and even hepatocellular carcinoma. Approximately 70-80% of patients develop chronic hepatitis, of which 20-30% progress onto liver cirrhosis.

As hepatocytes represent the primary site of HCV replication in vivo, the HCV genome has also been found in lymphoid cells. Infection of the lymphoid cells has been implicated in extra-hepatic manifestations of HCV infection such as mixed cryoglobulinemia and B-lymphocyte proliferative disorders.

Cellular Receptors for HCV

To date, the cellular receptor(s) for HCV remains controversial. The observations that HCV can infect both hepatic and lymphoid cells suggest that HCV may use different cellular receptors to access different cell types. However, the absence of an in vitro system that supports HCV replication and particle assembly has hampered studies to elucidate the early steps of HCV infection, i.e. virus binding and entry. Association of HCV virions with β-lipoproteins in plasma has raised the possibility that HCV may use the LDL receptor (LDL-R) for viral entry. Others have proposed that CD81, a cellular surface protein belonging to the tetraspanin protein superfamily, is the putative receptor for HCV, based on the interaction of CD81 with recombinant truncated E2 protein of HCV 1a. Nevertheless, several studies have shown that using the truncated E2 protein alone may not accurately reflect interaction of the HCV virion with cells. Both E1 and E2 glycoproteins are known to associate in two types of complexes: (i) heterodimers stabilized by non-covalent bonds, which presumably represents the pre-budding form of the viral envelope, and (ii) high molecular mass disulfide-bonded aggregates representing the misfolded proteins. Indeed, using a pseudotype vesicular stomatitis virus (VSV) expressing either HCV E1 or E2 protein, it has been shown that both proteins are required for efficient infection and fusion into target cells. Furthermore, the HCV virion binds to mononuclear cell lines regardless of their CD81 expression, while recombinant E2 protein binds poorly to cells that lack CD81.

Deficiencies

The structure of HCV virions has not yet been elucidated. This is in part due to the difficulties to obtain sufficient amounts of free, purified virion. So far, modeling of HCV ultrastructure is based on data obtained from other members of the Flaviviridae family (dengue and tick-borne encephalitis viruses). Several studies have shown that the genome of HCV is detected in association with other components in the serum: immunoglobulins and β-lipoproteins. Although antibodies recognizing envelope proteins have been detected in the serum, no demonstration is available on the presence of circulating envelope proteins. A recent report suggests the presence of core containing particles in the serum.

No HCV vaccine is yet available and the current treatment of chronic hepatitis (interferon in combination with ribavirin) is at best only effective in 61% of cases. Efficacy in fact depends in part on the genotype of the infecting HCV strain. The initial steps of HCV infection (binding and entry) that are critical for tissue tropism and hence pathogenesis, is poorly understood. Studies to elucidate this process have been hampered by the lack of robust cell culture systems or convenient small animal models that can support HCV infection. Therefore, there is a need for systems for producing and isolating HCV or HCV-like particles.

### SUMMARY OF THE INVENTION

The present invention relates to new methods for obtaining HCV complexes and HCV-like particles from cells, particularly insect cells, infected with recombinant baculoviruses encoding HCV structural genes. In one method, cells are lysed, preferably with digitonin in the presence of protease inhibitors. Polyethylene glycol is slowly added to the lysate, to provide a precipitate that comprises complexes of the HCV structural proteins associated with lipid vesicles or micelles and complexes comprising viral structural proteins in the form of insoluble aggregates. Preferably, the cells are thoroughly washed prior to lysis to remove recombinant baculoviruses in suspension in the culture medium. In another method, the lysate is centrifuged through a sucrose cushion, preferably a 20% sucrose cushion. Preferably, the pellet is then subjected to equilibrium ultracentrifugation, to provide a preparation of HCV-like particles. Preferably, the infected cells are thoroughly washed prior to lysis to remove baculovirus in suspension in the culture medium. The particles obtained by this method are heterogenous in size. Fractions containing viral structural proteins typically comprise three subpopulations of particles whose average diameters are about 35, 42, and 49 nm. The third method comprises subjecting the infected cells to hypertonic/hypotonic shock, and then lysing the cells with digitonin in the presence of protease inhibitors. Preferably, lysis and hypotonic shock are performed simultaneously. The lysate is pelleted, fractionated, preferably by equilibrium ultracentrifugation, to provide a population of homogenous HCV-like particles having an average diameter of about 50 nm. As used herein the term “homogenous” means that both the size and the shape of the particles are similar. Preferably the cells are washed thoroughly prior to hypertonic/hypotonic shock to remove recombinant baculoviruses in suspension in the culture medium. The present invention also relates to the preparations of HCV structural protein complexes and HCV-like particles obtained by the present isolation methods.

The invention also relates methods of using the HCV complexes and HCV-like particles as screening tools, diagnostic tools, and immunogenic compositions. In one embodiment, the present preparations, particularly the preparations of HCV-like particles, are used to detect specific anti-HCV antibodies in HCV-infected patients. In another embodiment, the preparations, particularly the preparations comprising HCV-like particles are used with cultured cells expressing receptors for HCV, to screen for compounds or substances that interfere with binding of the HCV-like particles to the cells and/or interfere with internalization of the HCV-like particles by the cells. In another embodiment, the HCV-like particles are used to identify cellular receptors for binding of the virus to cells. In another embodiment the preparations, including the HCV structural protein complexes and HCV-like particles, are used as immunogenic compositions to induce production of anti-HCV antibodies in a mammal, including humans.

Methods of treating HCV using the compounds or substances that interfere with binding or internalization, especially those that interfere with binding of the present HCV-like particles to asialoglycoprotein receptors, are also part of the invention.

### BRIEF DESCRIPTION OF THE DRAWINGS

The present invention may be more readily understood by reference to the following drawings wherein:

FIG. 1 is a schematic diagram of recombinant Bac-HCV.1a.S and Bac-HCV.1a.p1 constructs. Two recombinant baculoviruses encoding for the structural proteins HCV of 1a genotype (H77 strain): core, E1 and E2/p7 proteins (Bac-HCV-S) or that of lacking the p7⁻ protein (Bac.HCV-S/p7) were generated.

FIG. 2 is a characterization of HCV structural proteins. (A) Profile of HCV-SP after equilibrium sucrose gradient centrifugation. Insect cells were infected with recombinant Bac.HCV-S and were harvested at 3 days post-infection. HCV-S proteins were purified on an equilibrium sucrose gradient centrifugation. One-ml fractions were collected from the top and protein concentration was measured (squares, dotted line). 50 μl of each fraction was tested for E2 reactivity with AP33 mAb by ELISA (diamonds, full line). A similar pattern was observed for HCV-SP/p7⁻ (not shown). (B) Immunoblot analysis of HCV-SP.1a.S with anti-E2 and anti-core antibodies. 50 μl of each fraction was suspended into Laemmli buffer in denaturing conditions and analyzed with 10% SDS-PAGE, then blotted onto nitrocellulose membrane. HCV-SP was tested for E2 and core reactivity by incubating the membrane with AP33 and anti-core mAbs, respectively; antigen-Ab complexes were revealed by incubating the membrane with HRP-coupled anti-mouse antibody, then submitted to chemiluminescence reaction (ECL) and autoradiography. Antibody reactivity against both solubilized E2 and core is indicated with arrows, as well as reactivity against insoluble aggregates (ins. aggr.) on the top of the gel. The last lane (+) is a positive control with recombinant E2 and core proteins expressed in mammalian cells.

FIG. 3 is cell binding of HCV-SP and HCV-SP/p7⁻. (A) Binding of light and heavy fractions of HCV-SP to cells. Cells were incubated with HCV-SP derived from strain 1a of HCV; both light (open bars) and heavy (full bars) fractions were tested. Incubating cells with anti-E2 mAb followed by FITC-labeled goat anti-mouse IgG and submitting them to FACS analysis (FACscan) detected cell-bound HCV-SP. Nonspecific fluorescence was measured by adding primary and secondary antibodies in the absence of HCV-SP to cells. Cytotoxicity of both light (open triangles) and heavy (crosses) fractions was indirectly evaluated by the shift of cell size and granularity to the bottom left corner. (B) Binding of HCV-SP and HCV-SP/p7⁻ to HepG2 cells. Both light (full symbols) and heavy (open symbols) fractions obtained from Bac.HCV-S (squares) and Bac.HCV-S/p7⁻ (triangles) constructs were tested for cell binding that was measured as above.

FIG. 4 is binding of HCV-SP.1a.S to primary human hepatocytes, HepG2 and Molt-4 cells. Cells of various types were incubated with HCV-SP; cell-bound HCV-SP and nonspecific fluorescence were measured. The left panels represent the histogram pattern of HCV-SP binding to target cells. The right panels show the quantified results: a) in percentage of positive cells (diamonds): cells were considered positive when they displayed fluorescence with a value above that of the nonspecific fluorescence threshold; b) in mean fluorescence intensity (MFI; bars): MFI was determined for each cell after subtraction of nonspecific fluorescence value. The results presented were mean value obtained from three independent experiments.

FIG. 5 is HCV-SP binding to HepG2 cells, which is inhibited by ligands of asialoglycoprotein receptor (ASGP-R). (A) HCV-SP binding to HepG2 cells is calcium-dependent. Cells and HCV-SP were suspended either in binding buffer in the presence of CaCl₂ (full squares) or binding buffer containing 5 mM EGTA in the absence of CaCl₂ (open squares), and binding assay was performed as above. (B, C) Effect of ASGP-R ligands on HCV-SP binding to HepG2 cells. In the panel (B), cells were preincubated in binding buffer (with CaCl₂) at 4° C. in the presence of various concentrations of asialo-orosomucoid (ASOR), as indicated on the graph; then, binding assay was performed. In the panel (C), cells were preincubated at 4° C. with buffer alone (control) or with 1 mg/ml 19S-thyroglobulin (19S-Tg), 0.4 mg/ml asialo-thyroglobulin (asialo-Tg) or anti-ASGP-R peptide polyclonal antibody ( 1/100), pre-immune antibody had no effect (not shown), then with HCV-SP and binding assay was performed.

FIG. 6 is internalization of radio-labeled HCV-SP in HepG2 cells. Sf9 insect cells were infected with recombinant Bac.HCV-S baculovirus, then incubated with [³⁵S]-methionine-cysteine mix. HCV-SP was prepared, purified and radio-labeled material (50 μg/ml) was incubated with HepG2 cells at 37° C. for the indicated time. Cells were harvested, disrupted and submitted to cell fractionation. Four membrane fractions were isolated, each enriched in either plasma (full circles), microsomial/mitochondrion (fall squares), rough endoplasmic reticulum (open triangles) or smooth endoplasmic reticulum (full triangles) membranes. Radioactivity uptake was quantified by liquid scintillation counting.

FIG. 7 is co-localization of dye-labeled HCV-SP and ASGP-R GFP-hH1 in the nuclear envelope area. HepG2 cells expressing a fusion protein between GFP and ASGP-R hH1 subunit (GFP-hH1-HepG2 cells) were seeded into sterile glass 8-chamber slides one day before the assay. HCV-SP was dye (CM-DiI)-labeled and purified. GFP-hH1-HepG2 cells were incubated with 10 μg/ml CM DiI-labeled HCV-SP for 60 min at 37° C.; the cells were then rinsed, fixed with 4% paraformaldehyde and the slides were mounted with DAPI/antifade system and kept in the dark at 4° C. until they were analyzed by laser scanning confocal microscopy (LSCM) in both green (GFP; top right panel) and red (CM-DiI; top left panel) wavelength channels. Serial horizontal sections (from top to bottom: number 1 to 12) from a single cell obtained in both green and red wavelength channels (top panels) were superposed (bottom left panel); areas displaying co-localization (yellow color=green+red colors) are shown in the bottom right panel: threshold was applied to keep only most significant pixels; darkness increases with intensity of co-localized signals.

FIG. 8 is internalization of HCV-SP into GFP-hH1-transfected HepG2 cells. GFP-hH1-HepG2 cells were first incubated with 10 or 20 μg/ml dye labeled HCV-SP (top panels) or HCV-SP/p7⁻ (bottom panels), or without, as indicated on the figure, in serum-free medium at 4° C. for 30 min; this step was followed by further incubation at 37° C. for 60 min. The cells were then submitted to LSCM analysis in both green (GFP) and red (CM-DiI) wavelength channels; horizontal sections (6 per cell or group of cells) obtained in both green and red wavelength channels were superposed: areas displaying co-localization appear in yellow color.

FIG. 9 is binding of HCV-SP to ASGP-R transfected 3T3-L1 cells. Panel (A), mouse fibroblasts (3T3-L1 cells) were incubated with HCV-SP. Panel (B), 3T3-L1 cells were transfected to co-express two subunits of human liver ASGP-R (hH1 and hH2) and cell lines were established: clone 3T3-22Z co-expressed full length of both hH1 and hH2, whereas clone 3T3-24X co-expressed full-length hH1 with a variant of hH2 (hH2′) that has a truncated cytoplasmic domain (non-functional variant). Total RNA was extracted from parental 3T3-L1 cells (wt), from clones 3T3-22Z and 3T3-24X, or HepG2 cells and submitted to RT; these cDNAs were then used to amplify by PCR a DNA fragment corresponding to either ASGP-R hH1 or hH2 subunits, as indicated Panel (C), both 3T3-22Z (squares) and 3T3-24X (triangles) cells, as well as parental 3T3-L1 cells (circles), were challenged with various amounts (2.5-10 μg/ml) of either HCV-SP (open symbols) or HCV-SP/p7⁻ (full symbols) and incubated for 2 hrs at 4° C. Cell-bound HCV-S protein was detected by flow cytometry. Histograms of the binding of either HCV-SP (top panels, 4° C.) or HCV-SP/p7⁻ (middle, 4° C., and bottom, 37° C., panels) to 3T3-22Z (right panels) and 3T3-24X (left panels) cells are presented in panel (D).

FIG. 10 is internalization of labeled HCV-SP into ASGP-R transfected 3T3-L1 cells. Panel (A), ASGPR hH1/hH2-dual-transfected 3T3-L1 cells (clone 3T3-22Z=22Z+19 or clone 3T3-24X=24X+19) or wild-type 3T3-L1 cells (wt) were incubated in the presence of 10 μg/ml labeled HCV-SP or HCV-SP/p7⁻ for 30 min at 37° C. The cells were submitted to LSCM analysis in the red (CM-DiI) wavelength channel. Sections of two distinct cells are shown for each condition. Panel (B), 3T3-22Z cells were incubated with 10 μg/ml CM-DiI-labeled HCV-SP for 30 min at 37° C.; 15 sections were obtained after LSCM analysis of a single positive cell and are shown from the top (upper left picture) to the bottom (lower right picture).

FIG. 11 is characterization of HCV-LPs 1a. (A) HCV-LPs 1a were harvested on day 3 post-infection and purified. Eleven fractions (1 ml) were collected from the top and tested for E2 reactivity by ELISA. (B) Western blot analysis of HCV-LPs. The similar fractions collected from (A) were run on SDS-PAGE, followed by Western blot analysis with anti-E2 (ALP98), anti-E1 (A4) and anti-core (C1) mAbs. (C) Cryoelectron micrograph of HCV-LP 1a. Bar, 200 nm.

FIG. 12 is HCV-LPs binding to human hepatic and T cells. Binding of HCV-LPs to human hepatic (primary human hepatocytes, HepG2, HuH7, NKNT-3) and T (Molt4) cells was detected by anti-E2 mAb followed by FITC-labeled goat anti-mouse IgG (indirect method). x axis, the mean fluorescence intensity (MFI); y axis represents the number of cells. HCV-LPs did not bind to Aro, a human thyroid cell line.

FIG. 13 is characteristics of HCV-LP binding to cells. (A & B) Dose-dependent binding. Binding of HCV-LPs to PHH, HepG2, NKNT-3 and Molt-4 cells were analyzed. Nonspecific fluorescence was measured by adding primary and secondary antibodies to cells in the absence of HCV-LPs. The MFI was determined after subtracting nonspecific fluorescence value. Results presented are representative of three independent experiments. (C) Calcium-dependent binding. NKNT-3 cells and HCV-LPs were resuspended in 10 mM Tris-HCl, 150 mM NaCl buffer containing 5 mM EGTA, and the binding assay was performed. (D &E) Scatchard plot analysis of HCV-LPs binding. SYTO-labeled HCV-LPs (1-200 μg/ml) were incubated with cells for 1 h at 4° C. After washing, cell-bound HCV-LPs were analyzed by flow cytometry. Bound (B) and free (F) HCV-LPs for each concentration was determined based on the MFI of 100 μg/ml HCV-LPs in the absence of cells regarded as total input (T).

FIG. 14 is inhibition of HCV-LPs binding to cells by anti-E1 and -E2 antibodies. SYTO-labeled HCV-LPs were pre-incubated with 20-100 μg/ml of anti-E2 (AP33, ALP98), anti-E1 (A4), or isotype control IgG for 2 h at 4° C. The HCV-LPs-antibody mixtures were then incubated with Molt-4 cells for 1 h. Cell-bound HCV-LPs were analyzed. (A) Flow cytometry histogram of HCV-LPs binding in the presence (20 μg/ml) (open graph) and absence (black filled graph) of antibodies. Background binding is shown as the gray graph. (B) Dose response inhibition of HCV-LPs binding by the respective antibodies.

FIG. 15 is effect of CD81 on HCV-LP binding to cells. (A) Effect of human LEL-CD81 on HCV-LP binding. SYTO-labeled HCV-LPs were pre-incubated with increasing amounts of soluble human LEL-CD81 for 2 h at 4° C. prior to addition to Molt-4, NKNT-3 or HuH7 cells. The binding assay was performed. The top panel shows the flow histograms and the bottom the MFIs. (B) Effect of anti-CD81 on HCV-LP binding. Molt-4 and HuH7 cells were pre-incubated with mouse anti-human CD81 IgG (20 μg/ml) for 2 h at 4° C., then SYTO-labeled HCV-LPs were added and further incubated for 1 h at 4° C. Cell-bound HCV-LPs were analyzed.

FIG. 16 is effect of VLDL, LDL and HDL on HCV-LP binding to Molt-4 cells. Cell-bound HCV-LPs were analyzed by flow cytometry using indirect method (A & B) or direct method (C). (A) Increasing concentrations of HCV-LPs with or without LDL (0.5 mg/ml) were added simultaneously to cells. (B) Alternatively, HCV-LPs were pre-incubated with LDL for 2 h at 4° C. before added to cells. (C) SYTO-labeled HCV-LPs were incubated with cells for 1 h at 4° C. and cell-bound HCV-LPs were analyzed as described in M&M (open bar). Cells were pre-incubated with VLDL, LDL, HDL (0.5 mg/ml), or anti-human LDL-R IgG (20 μg/ml), for 2 h at 4° C., before addition of SYTO-labeled HCV-LPs (striped bar). Alternatively, SYTO-labeled HCV-LPs were pre-incubated with VLDL, LDL, or HDL at 4° C., before added to cells (closed bar).

FIG. 17 is confocal microscopy analysis of labeled-HCV-LPs internalization by cells. HuH-7 cells were incubated with CM-DiI labeled HCV-LPs at 4° C. (A) and then at 37° C. (B). As negative control, cells were incubated with CM-DiI labeled control Bac-GUS preparation at 37° C. (C). NKNT-3 cells were incubated with SYTO-labeled HCV-LPs at 4° C. (D) and then at 37° C. for 30 min (E). As negative control, cells were incubated with SYTO-labeled Bac-GUS at 37° C. for 30 min (F). NKNT-3 cells were incubated with SYTO-labeled HCV-LPs for 15 min at 37° C. (G). Alternatively, cells were incubated with SYTO-labeled HCV-LPs that had been pre-incubated with anti-E1/-E2 antibodies for 2 h (H). On each panel, six images represent the top to the bottom of cells (left to right) are shown.

FIG. 18 is a profile of new HCV-LP following equilibrium sucrose gradient centrifugation. (A) 10⁸ cells were grown in SF900 II medium (GIBCO BRL) and infected with Bac.HCV.1a.S at a multiplicity of infection (MOI) of 1 or 10 for 1 hr at 27° C. Without removing the inoculum, fresh medium containing 0.5% fetal bovine serum and antibiotics/antimycotic was added to reach a total volume of 30 ml. Cells were grown at 27° C. (125 rpm) and harvested at 2, 3, or 4 days post-infection. All purification steps were carried out on ice. The pellet was resuspended in TNC buffer, and applied onto a 20-60% sucrose gradient and centrifuged at 100,000-×g for 16 hours. Ten 500 μl-fractions were collected from the top of the tube. Fractions containing HCV-LP were stored at −70° C. Protein concentration was determined using Coomassie Plus protein assay reagent with BSA as the protein standard. (B) Fractions collected from (A) were analyzed by SDS-PAGE followed by Western Blot using specific anti-E2 (ALP98), anti-E1 (A4) and anti-core (C1) monoclonal antibodies. The figure shows fractions 3-9 of HCV-LP purified from insect cells 3 days post-infection with MOI 10.

FIG. 19 shows histograms of HCV-LP binding to human hepatic cells NKNT-3 (before and after transduction with AdCANCre, HuH7, and kidney (293) cells. Human hepatic cells (HuH7) and kidney cells (293) were obtained from American Type Culture Collection. An immortalized human hepatocytes (NKNT-3) and a replication-deficient recombinant adenovirus (Ad) that express the Cre recombinase tagged with a nuclear localization signal (AdCANCre) was used. Differentiation of NKNT-3 cells to mimic normal primary hepatocytes was achieved by transduction with AdCANCre followed by selection with G418 (Ad-NKNT-3). Cells were grown in Chee's Modified MEM containing 5% fetal bovine serum and were analyzed for HCV-LP binding at 3 days post-transduction. HCV-LP was directly labeled with SYTO-12 (nucleic acid dye) according to the manufacturer's protocol. Briefly, HCV-LP were incubated with 5 μM of SYTO-12 in TNC buffer at 4° C. for 15 min and re-purified through a 30% sucrose cushion to remove free dye. 2×10⁵ cells were incubated with 2.5 μg of SYTO 12-labeled HCV-LP in 50 μl TNC buffer containing 1% BSA and a cocktail of EDTA-free protease inhibitors, for 1 hr at 4° C. Cells were washed once with PBS, detached with 0.25 mM EDTA (in PBS) for 10 min at 37° C., and resuspended in binding buffer. After washing, cell-bound HCV-LP was analyzed by flow cytometry. For each cell type, histogram shown is cells in the absence of HCV-LP (gray graph) and after incubation with HCV-LP (black graph).

FIG. 20 shows the effect of anti-E2, anti-E1 and anti-core antibodies on HCV-LP binding to Ad-NKNT-3 cells. SYTO 12-labeled HCV-LP were pre-incubated with 20 μg/ml of anti-E2 (ALP98), anti-E1 (A4), or anti-C mAbs for 2 h at 4° C. and were then incubated with Ad-NKNT-3 cells for 1 h (open graph). As control, cells were incubated with HCV-LP in the absence of antibodies (closed graph). After washing, cell-bound HCV-LP was analyzed by flow cytometry.

FIG. 21 shows the effect of lipoproteins on HCV-LP binding to NKNT-3 and Ad-NKNT-3 cells. Cells were transduced with recombinant AdCANCre, and HCV-LP binding was performed at 3 days post-transduction. 2×10⁵ cells were incubated with 1.5 or 2.5 μg of SYTO 12-labeled HCV-LP (closed bar) for 1 hr at 4° C., and analyzed by flow cytometry. (A, B) NKNT-3 or Ad-NKNT-3 cells were pre-incubated with apolipoprotein E4 for 2 hr at 4° C. before adding HCV-LP and incubating for another 1 hr (striped bar). (C, D) Cells were pre-incubated with 0.5 mg/ml of LDL (hatched or striped bar) or without (closed bar), as a control, before adding dye-labeled HCV-LP. Alternatively, HCV-LP were pre-incubated with LDL before adding to cells (open bar). (E, F) Cells were pre-incubated with 0.5 mg/ml of HDL before adding dye-labeled HCV-LP (hatched bar); as a control, cells were incubated with HCV-LP in the absence of LDL (closed bar). Alternatively, HCV-LP were pre-incubated with HDL before adding to cells (open bar).

FIG. 22 shows the effect of AGSP-R ligands on HCV-LP binding to NKNT-3 and Ad-NKNT-3 cells. (A) Cells were pre-incubated with rabbit anti-ASGPR antibody for 2 hr at 4° C. before added with SYTO 12-labeled HCV-LP (striped bar). As control, cells were incubated with HCV-LP in the absence of anti-ASGP-R antibody (closed bar). (B) Cells were pre-incubated with 0.5 mg/ml of Tg 19S for 2 hr at 4° C. before SYTO 12-labeled HCV-LP was added (striped bar). Alternatively, HCV-LP were pre-incubated with Tg 19S for 2 hr at 4° C. before added to cells (open bar).

### DETAILED DESCRIPTION OF THE INVENTION

Because of the lack of in vitro systems for HCV replication and the inability to obtain the purified virus in sufficient quantity, virologists have attempted to express HCV genes in various expression systems with the idea that expressed HCV structural proteins would assemble into virion-like structures. It is well known for some viruses that expression of recombinant virus structural proteins in eukaryotic cells leads to the spontaneous-formation of pseudo-viral particles, so called viral- or virus-like particles.

In 1998, Baumert et al. reported that expression of recombinant structural proteins of HCV in insect cells led to the formation of virus-like particles, so-called hepatitis C viral-like particles (HCV-LP). The structural genes of HCV derived from 1b genotype were cloned into baculovirus allowing their expression under control of the polyhedrin promoter. These investigators also described a method of purifying HCV-LP from the infected insect cells. Insect cells were infected with an inoculum of recombinant baculovirus, in general, at a multiplicity of infection per cell of 1. Four days after infection, insect cells were harvested and lysed by sonication and homogenized in 50 mM Tris-HCl, pH 7.4, containing 50 mM NaCl, 0.5 mM EDTA, 0.1% NP40 and a cocktail of protease inhibitors. Cell lysates containing HCV-LP were centrifuged through a 30% sucrose cushion at 100,000-×g for 6 h. The pellet was homogenized and sonicated; the resuspended pellet then subjected to ultracentrifugation on a 20-60% sucrose gradient at 150,000×g for 22 h. Ten fractions were collected from the top of the tube and analyzed for the presence of HCV structural proteins by Western Blot.

The Baumert et al. method results in a low yield of HCV-LP. In addition, the HCV-LP resulting from this method is heterogeneous and contains significant amount of contaminating baculoviruses. This is a disadvantage, especially if the particles are to be used to immunize individuals, as the impurities present in the preparation might cause adverse immune reactions. The HCV-LP preparations that are obtained showed poor binding to target cells and significant death of those cells. This is unfortunate since a purified and biologically functional HCV-LP, theoretically, is a useful tool to identify cellular receptor(s) for HCV. Therefore, although HCV-LP are produced in cells infected with baculoviruses encoding HCV genes, isolation and purification of the particles from the infected cells has not yielded pure HCV-LP, and the quantities of HCV-LP obtained is not sufficient for significant further biological studies.

Purification and Characterization of Recombinant HCV Complexes and Particles From Infected Cells

The present invention describes new methods for purifying Hepatitis C recombinant material from insect cells infected with recombinant baculoviruses encoding HCV structural proteins. In one aspect the material is a complex of HCV structural proteins, referred to in the examples as “HCV-SP”. In another aspect, the material is HCV-like particles, referred to in the examples as “HCV-LP”.

In these methods, a protein expression system is used to express HCV structural proteins in eukaryotic cells. Preferably, the protein expression system used is a baculovirus expression system. One highly preferred expression system is a recombinant baculovirus whose genome comprises the structural genes of HCV. Preferably, the baculoviruses encode all of the structural proteins of HCV. In one embodiment, the recombinant baculovirus expresses core, E1 and E2-p7 proteins of HCV. In another embodiment, the recombinant baculovirus expresses core, E1 and E2, without p7. Transcription of the genes encoding the HCV proteins is driven by powerful promoters that initiate transcription of the HCV genes within the baculovirus after host cells are infected. The expression system may also comprise one baculovirus that encodes some of the structural proteins of HCV and that a second baculovirus that encodes the remaining HCV structural proteins. Methods for incorporating genes into the genome of baculoviruses are well known in the art. Such methods involve recombinant DNA technology and are well described in the art. Many such methods are described in U.S. Pat. No. 6,387,662 of Liang and Baumert.

Although it is possible to use any eukaryotic cell that has a glycosylation system, such as mammalian cells as a host cell for the expression system, it is highly preferred to use insect cells that are natural hosts or that are engineered to be hosts for baculovirus. There are a variety of methods known in the art for growing insect cells in culture and for infecting such cells with the baculoviruses. Any of these methods can be used.

Once insect cells are infected with the HCV structural protein encoding baculoviruses, the HCV proteins assemble into virus-like particles. Such particles can be detected within the baculovirus-infected cells by various methods, one being immunofluorescence using one or more antibodies specific for HCV proteins. Other methods for detecting the particles are available. The preferred method of choice is electron microscopy, which allows visualization of viral-like particles in the infected cells, preferably in combination with immunolabeling method.

Three different methods for purification of the HCV material from the host cells are described herein. For convenience, the methods described herein provide details of purification as they relate to baculovirus infected cells. The methods involve lysis of the infected cells in order to release viral protein complexes and/or virus-like particles from within the cells. The methods used for lysis preferably lyse the cells without damaging or by minimally damaging the virus-like particles within the cells. Preferably the cells are thoroughly washed prior to lysis or hypertonic shock, as described below, to remove recombinant baculoviruses in suspension in the culture medium.

In the first method of purification, cells containing HCV-like particles, e.g., baculovirus infected cells (Example 2) are lysed in a buffer comprising digitonin and protease inhibitors. Preferably, the concentration of digitonin used is less than 0.25%. Preferably insoluble debris is removed from the lysate, for example by centrifugation, and the resulting supernatant precipitated with the addition of polyethylene glycol (PEG). Various concentrations of PEG can be used, at various pHs and in various buffers, depending on the time and temperature of treatment. Good results have been obtained using PEG 8000 in 0.15 M NaCl at a concentration of 10%. Layering the precipitate onto a sucrose gradient, and subjecting the gradient to ultracentrifugation can achieve further purification. After a suitable time of centrifugation, fractions are collected from the sucrose gradient and tested for the presence of virus-like particles. This testing can be done in a variety of ways. One way is analysis of the proteins within the collected fractions by sodium dodecyl sulfate polyacrylamide gel electrophoresis (SDS-PAGE). This separation technique may be used as a prelude to Western blotting, where HCV proteins are detected using one or more antibodies specific for the proteins. Such methods are well known in the art. The preparation that results from this first method comprises complexes of the HCV structural proteins associated with lipid vesicles or micelles and complexes comprising viral structural proteins in the form of insoluble aggregates.

In the second method for virus-like particle purification (Example 11), cells containing HCV-like particles, e.g., baculovirus-infected cells are lysed with digitonin. Insoluble debris is removed by centrifugation and the supernatant is centrifuged over a sucrose cushion, e.g. a 20% or 30% sucrose cushion. The pellet is resuspended and, preferably layered onto a gradient and ultracentrifligation is performed. The gradient can be a sucrose gradient. Alternatively, the gradient can be of various types known in the art. For example, the gradient can comprise cesium chloride or other iodinated compounds, nycodenz or iodixanol for example. After a suitable time of centrifugation, fractions are collected from the gradient and tested for the presence of virus-like particles, as described above.

The third method for purification of virus-like particles (Example 18) involves hypertonic/hypotonic of cells containing HCV-like particles, e.g., baculovirus infected cells. The method suspending the cells in a hypertonic buffer (e.g., Hepes plus glycerol), then in a hypotonic buffer (e.g., Hepes). It is also possible to use other components or steps to achieve successive treatment in a hypertonic buffer and a hypotonic buffer. For example, sucrose or hypertonic saline solution can be followed by hypotonic shock. Preferably, lysis and hypotonic shock are performed simultaneously. Insoluble debris is removed from the lysate preferably by centrifugation and the supernatant is centrifuged over a sucrose cushion, e.g. a 20% or 30% sucrose cushion to provide a preparation of HCV-like particles that are approximately 50 nm in size.

The virus-like particles obtained from the methods of purification described above are characterized. Such particles contain one or more, preferably all, of the HCV proteins expressed in the baculovirus-infected insect cells. Such particles also contain lipids. Such particles may or may not contain nucleic acid. Nucleic acid contained in the particles is preferably RNA.

There are a variety of methods well known in the art of virology for characterizing virus particles. SDS-PAGE with or without Western blotting has already been described. Other immunological methods, ELISA for example, can also be used to detect and analyze proteins present within or associated with the virus-like particles. Electron microscopy can be used to visualize and to measure the size of the particles. Cryoelectron microscopy can be used. Ultracentrifugation can be used to determine buoyant density of the particles. Other methods can be used to detect and analyze a viral genome that may be present within the particles. Such a method, for example, consists in extracting RNA from virus-like particles and subjecting the extract to a step of reverse transcription to synthesize cDNA. The HCV specific DNA fragment is then amplified using specific primers and thermoresistant DNA polymerase (polymerase chain reaction, or PCR), followed by agarose gel electrophoresis and ethidium bromide staining to visualize the size of viral specific DNA fragment.

Other assays may be used to ascertain various functions of the particles. For example, assays to determine whether the virus-like particles bind to host or target cells can be used. The same or other assays can be used to determine whether the particles enter host or target cells. Some such assays are described in various Examples of this application.

In addition there are many techniques and methods that exist in the art of virology that can be used to detect and measure various aspects of viruses or virus particles or their functioning or interaction with cells. Such methods are well known in the art and can be found in numerous textbooks or laboratory manuals of virology. Such methods can be used to analyze and test the virus-like particles of the present invention and their functioning.

Briefly, the characteristics of the HCV recombinant material obtained by the three different methods of purification described above are as follows. The material resulting from the first purification method described above are complexes of HCV structural proteins associated with lipid vesicles or micelles or complexes that are aggregates of the HCV structural proteins. The material resulting from the second purification method described above are irregular particles containing E1-E2 envelope proteins representing three subpopulations of particles that are more apparent. The material resulting from the third purification method described above is a preparation of particles that are substantially homogeneous. As used herein the term “substantially homogenous means that the particles are similar in shape and vary in size by ±10% or less. The HCV-like particles prepared by this third purification method are approximately 50 nm (±10%) in diameter with an apparent structure resembling other known viruses of the family Flaviviridae. This latter method of purification preserves the structure of the virus-like particles during the purification process. Other characteristics of the HCV-like particles obtained from the three different methods of purification are described in the Examples of this application.

In those cases where the cells are thoroughly washed prior to lysis or hypertonic shock, the preparations contain very low levels of baculovirus particles. In the prior art methods discussed above, the % of baculovirus in the preparation is greater than 50% and can be as much as 80%. In the present methods the percent of baculovirus in the preparation is less than 30%. For example, method 1 results in the production of preparations that contain less than 30% and in some cases no baculovirus. Method 2 results in preparations that are expected to contain less than 10% baculovirus. Method 3 results in preparations that are highly purified and that typically contain 1% or less of contaminating baculovirus.

Assays

The HCV-like particles are used in variety of assays. In one type of assay, the particles are used to detect HCV in a sample, in the blood of a patient for example. In another type of assay, the HCV-like particles are used in assays to screen for compounds or substances that interfere or prevent binding of the particles to cells and/or internalization of the particles into the cell. In another type of assay, the present HCV-like particles can be used to detect and identify receptors or co-receptors for HCV.

A. Diagnostic Assays

Assays to detect HCV in a sample or to determine if an individual is or has been exposed or infected with the virus can be of a variety of types. One type of involves detecting antibodies in a subject that are cross-reactive with the HCV-like particles produced by the present invention. Many such assays are well known in the art. For example, such assays include competitive binding assays, direct and indirect sandwich-type immunoassays, agglutination assays and precipitation assays.

Because the HCV-like particles structurally mimic hepatitis C virions, the particles can be used to capture anti-HCV antibodies and antibodies that recognize the HCV-like particles can also recognize HCV. Generally, diagnostic kits using immunoassay formats use the HCV-like particles to assay for anti-HCV antibodies in a human infected with HCV, or use antibodies that bind to HCV-like particles to detect HCV in human tissue (such as blood or serum) obtained from an HCV-infected individual. The detection can be direct or indirect as is well known in the art.

Cell-free assays can be used to measure the binding of human antibodies in serum to HCV-like particles. For example, the particles can be attached to a solid support such as a plate or sheet-like material and binding of anti-HCV antibodies to the immobilized HCV-like particles can be detected by using a labeled anti-human immunoglobulin to visualize the bound anti-HCV antibodies attached to the HCV-like particles on the support. Similarly, the virus-like particles can be attached to inert particles such as latex beads, which can be used to detect human anti-HCV antibodies by detecting agglutination or capture of the particles at a discrete position.

In another type of assay, which can be used to detect either antibodies against HCV, or HCV particles in a sample, binding of the HCV-like particles to a cell to which HCV or HCV-like particles normally bind is used as the endpoint. In one embodiment, cultured cells to which HCV-like particles are capable of binding are used. Serum from a patient suspected of having antibodies specific for HCV is incubated with the HCV-like particles. The serum-incubated particles are then incubated with the cultured cells and it is determined whether the virus-like particles are able to bind to the cells. If the patient serum contains antibodies specific for HCV, the antibodies bind to or inactivate the HCV-like particles. In such case, no binding of the HCV-like particles to the cells is detected. In the control study, where the HCV-like particles were not pre-incubated with patient serum, the particles bind to the cells.

In another embodiment of this assay, a patient sample suspected of containing HCV is incubated with the cultured cells to which HCV-like particles are capable of binding. Subsequently, HCV-like particles are incubated with the cells and it is determined whether the particles bind to the cells. In the case where the patient sample contains HCV, the HCV binds to the cells and inhibits binding of the HCV-like particles.

Binding of the HCV-like particles to the cells in assays as described above can be detected and quantified in a variety of ways. In one technique, the particles may be labeled using radioactive or nonradioactive labels. The label may be directly or indirectly coupled to the particles using methods well known in the art. For example, HCV-like particles may be radioactively labeled with ³H, ¹²⁵I, ³⁵S, ¹⁴C or ³²P using standard in vivo or in vitro labeling methods and the binding of HCV-like particles to cells may be detected using autoradiography or scintillation counting. The particles may also be labeled with labels that are non-radioactive. One such non-radioactive label attaches to the lipids of the virus envelope. The CellTracker dyes from Molecular Probes are of this type. Another type of dye binds to the nucleic acid of the particle. Examples of dyes of this type are the SYTO dyes, also available commercially from Molecular Probes.

B. Screening Tools.

Assays to screen for compounds or substances that interfere or prevent binding of HCV and HCV-like particles to cells can be of a variety of types. The HCV-like particles can be used to assay for proteins, antibodies or other compounds capable of inhibiting interaction between HCV and mammalian cells. For example, compounds that interfere with the ability of HCV to effectively infect human cells can be detected by measuring the ability of labeled HCV-like particles to bind to human cells, in vivo or in vitro, in the presence of the compound compared to control conditions where the compound is not present. Cell lines used in such assays have receptors for binding of HCV. Exemplary cell lines for detecting such interference with HCV-like particles include Hep 3B, HepG2, Chang liver, Daudi and MOLT-4, all available from the American Type Culture Collection (Rockville, Md.), and HuH7 cells, available from many research laboratories. Other such cell lines are primary human hepatocytes.

Cells that do not have receptors for HCV can also be used if the cells are manipulated in such a way that the cells express the receptors. Such cells that do not have receptors are 3T3-L1 cells, for example. One method for manipulating cells that do not express receptors is to transfect or otherwise introduce into the cells and express therein a nucleotide or nucleotide sequences that encode such receptors. Such nucleotide sequences are, for example, cDNAs from human liver encoding the hH1 and hH2 of the asialoglycoprotein receptor (ASGP-R).

Purified HCV-like particles are incubated with the cells and it is determined if the particles bind to the cells. Binding can be determined in a variety of ways. One way to determine binding is to label the HCV-like particles. The particles can be radioactively labeled or can be non-radioactively. In the case of radioactively labeled particles, binding of the particles to cells can be detected by autoradiography or scintillation counting of the cells. In the case of non-radioactively labeled particles, for example in the case where the particles have been labeled with a fluorochrome, fluorescence imaging of the cells will detect the attached particles. Alternatively, the fluorochrome-labeled particles can be detected after flow cytometry analysis of the cells. Binding of the HCV-like particles to the cells can also be detected in the case that the particles are not labeled. In this case, particles bound to cells can be detected by incubating the cells and attached particles with an antibody reactive with the virus. If the antibody is labeled with a fluorochrome or reacted with a second antibody that is labeled with a fluorochrome, attached particles can be detected after imaging or flow cytometry analysis of the cells.

Using such a cell binding assay, compounds or substances suspected of interfering with the binding of HCV-like particles and HCV to cells, is detected by first incubating the substance with either or both of the cells or the HCV-like particles, then incubating the cells with the particles and assaying for binding of the particles to the cells. Compounds or substances that interfere with particle binding will cause a reduction in the measurement of particles bound to the cells as compared to controls where no compound or substance was added before the cells and particles were contacted with one another.

Cell lines have been created that are readily used as assay targets for HCV infection. To do this, a nucleotide sequence encoding a receptor for HCV is introduced into cultured cells. For example, nucleotide sequences encoding subunits of ASGP-R are transfected into cells. The cells can be cells that have no receptors (e.g., 3T3-L1 fibroblasts) or can be cells that express receptors (e.g., HepG2 cells). In the latter instance, the level of receptors on the cell surface is much higher in transfected cells than in non-transfected cells. Preferably, the cells are also expressing a marker gene, which is linked to a promoter that is inducible upon virus entry or HCV-like particles internalization. Such cells are called indicator cells.

For example, HCV that bind the viral receptors on the surface of the indicator cells will cause induction of transcription of the marker gene, luciferase or green fluorescent protein (GFP). Induction of the marker gene is conveniently detected. Prior incubation of indicator cells with HCV-like particles will prevent the induction of transcription of the marker gene by the virus. Such indicator cells can be used to assay for HCV in fluid samples from a patient.

Such cells can also be used to assay for antibodies reactive with HCV in fluid samples of a patient. For example, HCV-like particles are contacted incubated with the fluid sample. Antibodies therein that are reactive with HCV, bind to the HCV-like particles and inactivate them. Subsequent contact incubation of the fluid-treated HCV-like particles with the indicator cells do not cause induction of expression of the marker gene to the same extent as contact with the cells of HCV-like particles that have not been contacted incubated by patient fluid not containing HCV-reactive antibodies.

Similarly, the indicator cells, along with the HCV-like particles, can be used to screen various substances and compounds for the ability to inhibit binding of HCV to a cell and/or to inhibit internalization of HCV into a cell. To do this, the indicator cells and/or HCV-like particles are incubated with a desired substance or compound. Subsequently, the HCV-like particles are incubated with the indicator cells and the level of induction of the marker gene is measured. Substances or compounds that inhibit HCV binding to the cells and/or internalization of HCV by the cells, will cause a reduction in the expression level of the marker gene of the indicator cells as compared to a similar control experiment where no substance or compound was used.

Similarly, antibodies that interfere with HCV infection of human cells can be detected and their ability to block infection can be measured by assaying the level of interaction between HCV-like particles and human cells (such as hepatocytes and HuH-7 cells) in the presence of the antibodies compared to the level of interaction achieved when the antibodies are absent.

Another type of assay can be used to measure internalization of virus by cells. In such an assay, virus is detected within cells. One method of doing this is by labeling HCV-like particles. The particles may be labeled by any of the methods described above. The labeled particles are incubated with cells and, at some later time, the cells are examined to determine if labeled virus or virus components can be detected within the cell. For example, in the case where radioactively labeled HCV-like particles are used, autoradiography of intact cells can be used to detect internalization. Another method is fractionation of various cell components or compartments using cell biological and/or biochemical techniques that are well known in the art. After the cell components are fractionated, scintillation counting is used to detect the radioactive label and determine if the virus has been internalized by the cell and where within the cell the HCV-like particles is located. In the case where HCV-like particles are not radioactively labeled, but, for example, are labeled with some type of fluorochrome as described earlier, cells can be fixed and then examined using methods such as confocal microscopy and flow cytometry.

The invention also encompasses methods of treating HCV infection in a patient using compounds or substances identified through use of the above assays, that inhibit binding of HCV to cells and/or inhibit internalization of HCV into target cells. Some such compounds or substances bind to ASGP-R or prevent binding of HCV to ASGP-R. Some such substances that have been identified include asialo-orosomucoid, thyroglobulin, asialo-thyroglobulin and antibodies reactive against peptides in the ASGP-R, such antibodies are preferably humanized antibodies. One specific antibody reactive against ASGP-R is a polyclonal antibody specific for a peptide of the CRD of hH1 subunit of the ASGP-R. Such compounds and substances can be used therapeutically to treat individuals infected with HCV or even prophylactically to prevent infection of individuals by HCV. The compounds and substances used in these methods are prepared into pharmaceutically acceptable compositions and easily administered to individuals at dosages that are therapeutically effective.

Compositions Containing HCV Structural Protein Complexes and HCV-Like Particles for Induction of an Immune Response

It should also be recognized that the HCV-like particles and the HCV structural protein complexes of the present invention could be used as an immunogenic composition to induce production of antibodies reactive with HCV in an animal. Such antibodies can be used in a variety of ways. One such use is to detect HCV in a sample from a patient in a diagnostic assay, many of which are known in the art. The anti-HCV antibodies can be made by a variety of methods that are well known in the art. In one such method, the HCV-like particles are injected into an animal, a rabbit, mouse, rat, rabbit, goat, sheep or horse, for example, to cause the animal to have a humoral immune response. In such animals, the serum contains antibodies specific for HCV. Antibodies can be used to detect HCV in patient samples.

In another method, HCV-like particles are used to make monoclonal antibodies, using methods well known in the art. Monoclonal antibodies that bind to HCV-like particles can readily be produced by fusing lymphatic cells isolated from an immunized animal using well-known techniques. Polyclonal or monoclonal antibodies that bind to HCV-like particles may be bound to a variety of solid supports such as polysaccharide polymers, filter paper, nitrocellulose membranes or beads made of polyethylene, polystyrene, polypropylene or other suitable plastics.

Vaccination against and treatment of HCV infection may be accomplished using pharmaceutical compositions, including HCV-like particles and HCV structural protein complexes. Suitable formulations for delivery of HCV-like particles are found in Remington's Pharmaceutical Sciences, 17th ed. (Mack Publishing Co., Philadelphia, Pa., 1985). These pharmaceutical compositions are suitable for use in a variety of drug delivery systems (Langer, Science 249:1527-1533, 1990).

HCV-like particles in compositions are suitable for single administration or in a series of inoculations (e.g., an initial immunization followed by subsequent inoculations to boost the anti-HCV immune response). The pharmaceutical compositions are intended for parenteral, or oral administration. Parenteral administration is preferably by intravenous, subcutaneous, intradermal, intraperitoneal or intramuscular administration. Parenteral administration may be preferentially directed to the patient's liver such as by catheterization to hepatic arteries or into a bile duct. For parenteral administration, the compositions can include HCV-like particles suspended in a suitable sterile carrier such as water, aqueous buffer, 0.4% saline solution, 0.3% glycine, hyaluronic acid or emulsions of nontoxic nonionic surfactants as is well known in the art. The compositions may further include substances to approximate physiological conditions such a buffering agents and wetting agents such as NaCl, KCl, CaCl₂, sodium acetate and sodium lactate. Aqueous suspensions of HCV-like particles can be lyophilized for storage and can be suitably recombined with sterile water before administration.

Solid compositions including HCV-like particles in conventional nontoxic solid carriers such as, for example, glucose, sucrose mannitol, sorbitol, lactose, starch, magnesium stearate, cellulose or cellulose derivatives, sodium carbonate and magnesium carbonate. For oral administration of solid compositions, the HCV-like particles preferably comprise 10% to 95%, and more preferably 25% to 75% of the composition.

HCV-Hike particles can also be administered in an aerosol such as for pulmonary and/or intranasal delivery. The HCV-like particles are preferably formulated with a nontoxic surfactant (e.g., esters or partial esters of C6 to C22 fatty acids or natural glycerides) and a propellant. Additional carriers such as lecithin may be included to facilitate intranasal delivery.

HCV-like particles can be used prophylactically as a vaccine to prevent HCV infection. A vaccine containing HCV-like particles contains an immunogenically effective amount of the particles in a pharmaceutically acceptable carrier such as those described above. The vaccine may further include carriers known in the art such as, for example, thyroglobulin, albumin, tetanus toxoid, polyamino acids such as polymers of D-lysine and D-glutamate, inactivated influenza virus and hepatitis B recombinant protein(s). The vaccine may also include any well-known adjuvant such as alum, aluminum phosphate and aluminum hydroxide. Double-stranded nucleotide or polynucleotides can also be used as adjuvants. When double-stranded polynucleotides are used as antigens, the vaccine preparation is preferably administered to the individual by intramuscular injection. The immune response generated to the HCV-like particles may include generation of anti-HCV antibodies and/or generation of a cellular immune response (e.g., activation of cytotoxic T lymphocytes or CTL) against cells that present peptides derived from HCV.

Vaccine compositions containing HCV-like particles are administered to a patient to elicit protective immune response against HCV, which is defined as an immune response that prevents infection or inhibits the spread of infection from cell to cell after an initial exposure to the virus. An amount of HCV-like particles sufficient to elicit a protective immune response is defined as an immunogenically effective dose. An immunogenically effective dose will vary depending on the composition of the vaccine (e.g., containing adjuvant or not), the route of administration, the weight and general health of the patient and the judgment of the prescribing health care provider. For initial vaccination, the general range of HCV-like particles in the administered vaccine is about 100 μg to about 1 gm per 70 kg patient; subsequent inoculations to boost the immune response include HCV-like particles in the range of 100 μg to about 1 gm per 70 kg patient. A single or multiple boosting immunizations are administered over a period of about two weeks to about six months from the initial vaccination. The prescribing health care provider may determine the number and timing of booster immunizations based on well known immunization protocols and the individual patient's response to the immunizations (e.g., as monitored by assaying for anti-HCV antibodies or to avoid hyperimmune responses).

For treatment of a patient infected with HCV, the amount of HCV-like particles to be delivered will vary with the method of delivery, the number of administrations and the state of the person receiving the composition (e.g., age, weight, severity of HCV infection, active or chronic status of HCV infection and general state of health). Before therapeutic administration, the patient will already have been diagnosed as HCV-infected and may or may not be symptomatic. A therapeutically effective dose of HCV-like particles is defined as the amount of HCV-like particles needed to inhibit spread of HCV (e.g., to limit a chronic infection) and thus partially cure or arrest symptoms or prevent further deterioration of liver tissue.

In one embodiment, HCV-like particles are used to immunize animal generally using a procedure where about 10 to 100 μg, preferably about 50 μg of the particles are initially administered to the animal to induce a primary immune response followed by one to about five booster injections of about 10 to 100 μg of HCV-like particles over a period of about two weeks to twelve months. Depending on the size of the animal to which the particles are administered, the dosage may vary, as will be readily determined by those skilled in the art. The timing and dosage of the booster injections in particular are determined based on the immune response detected in the animal, using methods well known to those skilled in the art. The virus-like particles are preferably administered subcutaneously as a suspension that includes an adjuvant such as Freund's complete or incomplete adjuvant, although a wide variety of available adjuvants are also suitable.

Another type of pharmaceutical composition that can be administered for the purpose of stimulating a protective immune response against HCV is a composition comprising HCV-like particles and cells, preferably cells that are antigen presenting cells. In one embodiment, dendritic cells are isolated from an individual and incubated with HCV-like particles. The dendritic cells internalize the HCV-like particles. The dendritic cells that have been incubated with HCV-like particles are then administered to an individual as part of a pharmaceutical composition, for the purpose of stimulating an immune response in the individual that is protective or therapeutic for HCV infection. Although dendritic cells can be used in this procedure, other types of antigen presenting cells can be used. It is also possible to take cells that are not antigen presenting cells, and express within those cells, increased levels of MHC class I and/or MHC class II molecules. Such cells are also made to express, on the cell surface, molecules to which an immune response is desired, HCV proteins for example. Such cells, expressing both MHC and the desired antigen, are used as a component of the pharmaceutical composition comprising the vaccine. This procedure is advantageous in that the previously described immunization procedures, in which HCV-like particles alone (no cells) comprise the vaccine, is that such procedures usually induce immune responses to dominant antigens, which are not always the protective antigens important for host defense.

In another embodiment, immunization is performed using a pharmaceutical composition made as follows: monocytes are isolated from an individual, transfected with double-stranded DNA and one or more genes encoding HCV proteins. The cells are then treated with mitomycin C, or other treatment to kμl the cells, and administered back into the individual, preferably by intramuscular or intraperitoneal injection.

In another embodiment, immunization is performed using a pharmaceutical composition made as follows: monocytes are isolated from an individual, transfected with a polynucleotide sequence encoding ASGP-R. These cells are then exposed to HCV proteins, which bind to and are internalized by the cells. These cells are then treated with mitomycin C and administered back into the individual as above.

In any of the embodiments where the pharmaceutical composition used for the vaccine comprises cells and HCV proteins, the cells can be incubated with one or more cytokines before administration into the individual for the purpose of providing cells that are better able to stimulate an immune response when administered to the individual.

In addition, one or more of the above compositions may be combined to provide an effective pharmaceutical composition to be used for immunization against HCV.

### EXAMPLES

The invention may be better understood by reference to the following examples, which serve to illustrate but not to limit the present invention.

### Example 1

### Baculoviruses Expressing HCV Proteins

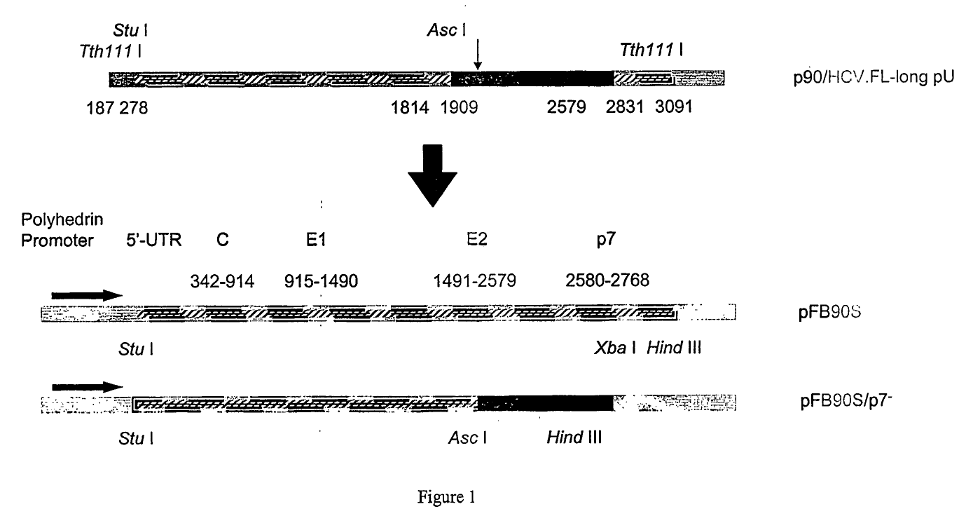

Two recombinant baculoviruses expressing the structural proteins of HCV derived from 1a genotype (H77 strain) were generated (FIG. 1). These two constructs express core, E1 and E2-p7 or core, E1 and E2 without p7.

A plasmid containing an infectious HCV clone of the 1a genotype H77 strain, p90/HCV.FL-long pU (gift of M. E. Major & S. M. Feinstone; FDA; Bethesda, Md.), was used as a template to generate two recombinant baculoviruses coding for the structural HCV proteins: core, E1 and either E2/p7⁺ (Bac.HCV-S) or E2/p7⁻ (Bac.HCV-S/p7⁻). The Bac.HCV.S has an additional 63 nt of the amino terminal part of NS2. This plasmid was digested with Stu I and Tth111 I, releasing a DNA fragment (nt 278-2831) corresponding to core, E1 and E2/p7⁺ proteins, which was subcloned between the Stu I-Xba I sites of a pFastBac plasmid, allowing its expression under the control of a polyhedrin promoter (pFB90S). A second DNA fragment (nt 1814-2579) was generated from p90/HCV.FL-long pU; PCR was performed with Pfu DNA polymerase and the two following primers 5′-AAG ACC TTG TGG CAT TGT GC-3′ (sense) and 5′-TCG AAA GCT TAC GCC TCC GCT TGG GAT ATG AGT-3′ (anti-sense); for construct purpose, a Hind III site (underlined) was introduced in this amplimer. The 775-bp PCR product was subcloned into the Sma I site (blunt-end) of pUC19 vector (pUC775). pUC775 and pFB90S plasmids were digested with Asc I and Hind III, respectively, to obtain a 671-bp DNA fragment (nt 1909-2579) and to remove a fragment (nt 1909-2831) of pFB90S. The 671-bp fragment was then ligated with the truncated plasmid (pFB90S/p7⁻) that encodes for an E2/p7⁻ protein. The schematic diagram of the cloning procedures is shown in FIG. 1. The nucleotide sequences of the recombinant baculoviruses were verified by restriction enzyme analysis and DNA sequencing.

Plasmids pFB90S and pFB90S/p7⁻ were used to generate recombinant baculoviruses, Bac.HCV-S and Bac.HCV-S/p7⁻, respectively, using BAC-to-BAC Baculovirus Expression System (Gibco-BRL/Life Technologies, Gaithersburg, Md.) according to the manufacturer's protocols. Virus titer was determined by BacPAK Baculovirus Rapid titer kit (Clontech, Palo Alto, Calif.).

Expression of core, E1, and E2 proteins of the recombinant baculoviruses in Sf9 cells (from Spodoptera fugiperda) was analyzed by indirect immunofluorescence. Indirect immunofluorescence was performed as follows: cells were seeded in a flat bottom 96-well plate (Sf9 cells attach after 1 h at 27° C. without shaking). When attached, the culture medium was removed and washed once with ice-cold PBS×1. Cells were fixed on ice with freshly prepared ice-cold methanol/acetone (50:50) for 2 min; fixation solution was then removed and washed 3 times with ice-cold PBS×1. Cells were incubated with PBS×1 containing 0.25% Igepal CA-630 (or NP-40) for 15 min on ice; detergent solution was removed and washed 3 times with ice-cold PBS×1. Cells were incubated with PBS×1 containing primary antibody ( 1/100) plus 0.1% Tween-1% BSA and 0.02% sodium azide for 1 h at room temperature with gentle shaking. Cells were washed 3 times with PBS×1 and incubated in the dark with FITC-coupled goat anti-mouse, antibody ( 1/250) in the same buffer for 45 min. The cells were washed 3 times with PBS×1 and analyzed with a fluorescence microscope.

### Example 2

### First Method of Purification—HCV Structural Proteins (HCV-SP)

Sf9 cells were grown at 27° C. in Sf900 medium (Gibco-BRL/Life Technologies, Gaithersburg, Md.) and were infected with recombinant baculovirus at multiplicity of infection (MOI) of 5 in a 500-ml Erlenmeyer flask, and cells were harvested at 3 days post-infection. All purification steps were carried out at 4° C. on ice. Cells were harvested (3,000 rpm for 15 min), washed once in 10 mM Tris-HCl pH 7.4, 150 mM NaCl, 1 mM CaCl₂ (TNC) buffer containing 1 mM Pefabloc SC and a cocktail of EDTA-free protease inhibitors (Roche, Indianapolis, Ind.), and finally resuspended at 1×10⁷ cells/ml in TNC buffer containing 0.25% digitonin and protease inhibitors (cf. above). Cells were homogenized, and placed on ice for 4 hr with gentle agitation, and centrifuged at 30,000×g for 45 min. The supernatant was collected, precipitated with 10% PEG 8000 and 0.15 M NaCl for 2 hr, and pelleted at 10,000 rpm for 30 min at 4° C. The pellet was resuspended in TNC buffer and briefly homogenized. 100-200 μl of homogenized suspension was applied onto a 10.5 ml of 20-60% sucrose gradient and centrifuged at 156,000×g for 16 hr. Fractions, 1 ml, were collected from the top of the tube and were tested for E1, E2 and core proteins by ELISA and western blot. Fractions containing Bac.HCV-S proteins (HCV-SP) were pooled, diluted with TNC buffer and pelleted at 100,000×g for 3 hr. Pellets containing HCV-SP were resuspended in TNC buffer and stored at −70° C. Protein concentration was determined using Coomassie Plus protein assay reagent (Pierce, Rockford, Ill.) with BSA as the protein standard. Similar methods were used to express and purify proteins produced with Bac.HCV-S/p7⁻ (HCV-SP/p7⁻).

### Example 3

### Characterization of HCV-SPs

The fractions collected from the sucrose gradients, as described in Example 3, were analyzed for the presence of E1, E2 and core proteins by both ELISA and Western blot. E2 ELISA was performed as described: a 96-well plate was coated with 100 μl (20 μg/ml in PBS) of GNA (lectin from Galanthus nivalis) at 37° C. for 3 hr. To prevent non-specific binding, 150 μl of 4% goat serum (in 5% skim milk-PBS) was added and incubated for 3 hr at room temperature. Samples containg HCV-SPs were diluted in 5% skim milk-PBS, added to each well and incubated at 4° C., overnight. Anti-E2 monoclonal antibody (mAb AP33, 100 μl, 6 μg/ml) was added and plate was incubated for 3 hr at 37° C. Peroxidase labeled goat anti-mouse IgG (at a dilution of 1/1000) was then added and incubated for 1 hr at 37° C. Bound antibodies were detected by adding ABTS Microwell Peroxidase Substrate System and measured on an ELISA reader at an optical density of 405 nm (OD 405 nm). Plate was washed six times with PBS between each step and, after addition of anti-E2 mAb, with PBS-0.05% Tween 20. All dilutions were made in PBS containing 5% skim milk.

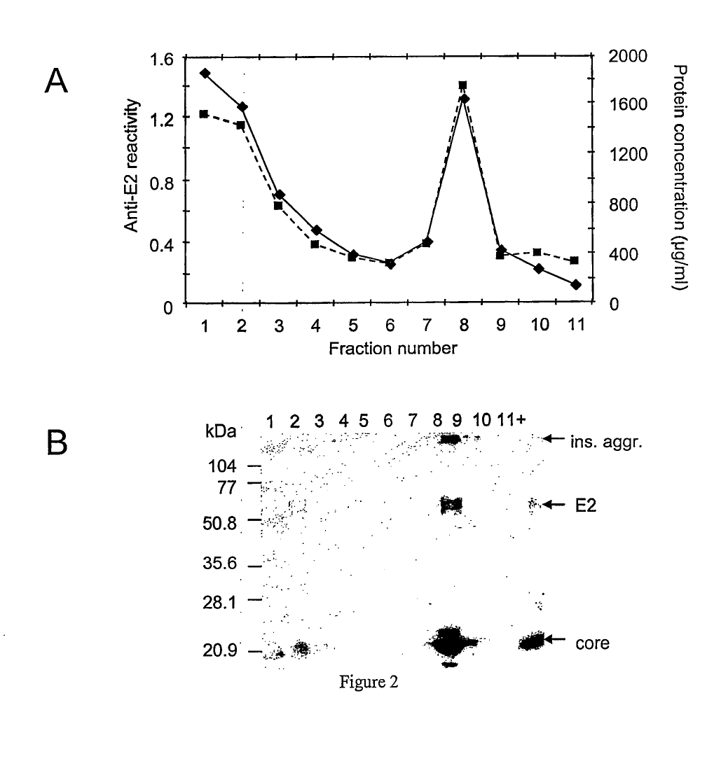

ELISA results (FIG. 2A) showed E2 reactivity was detected in two peaks: the lighter density (fractions 1-3) correspond to a buoyant density of 1.14-1.18 g/ml, and heavier density (fractions 8-9) correspond to buoyant densities of 1.2-1.25 g/ml. Western blot analysis (FIG. 2B) using anti-E2 mAb (ALP98) showed a group of major E2 protein bands of ˜70 kDa; the core protein was detected as a band at ˜20 kDa. Two major forms of E1 (˜33 and ˜28 kDa) that reflect the different extent of N-linked glycosylation were also observed (not shown). The E2 protein of HCV-SPs was recognized by conformation-sensitive anti-E2, H2 and H53 mAbs, indicating that the E2 protein of HCV-SPs assume a proper conformation.

### Example 4

### Cell Binding of HCV-SP and HCV-SP/p7⁻

Binding of the HCV-SP preparations to HepG2 cells was performed as follows: the assays were performed in a U-bottom 96-well plate. All the incubation (on a rocking platform) and centrifugation/washing steps (800 rpm, 5 min) were carried out at 4° C. All dilutions were made in ice-cold binding buffer (TNC buffer containing 1% BSA and a cocktail of EDTA-free protease inhibitors). Adherent cells (HepG2) were washed twice with PBS and detached with 2.5 mM EDTA (in PBS) at 37° C. for 10 min prior to use. Cells were rinsed once, resuspended in TNC buffer at 2×10⁶ cells/ml and 100 μl were added to each well. Bac.HCV-SP binding was measured by indirect labeling. 0.125-2.5 μg of HCV-SPs were incubated with cells for 2 hr, and cells were washed twice to remove unbound proteins. Anti-E2 mAb (AP33) was added and cells were incubated for 1 hr, washed twice, and further incubated for 1 hr with FITC goat anti-mouse IgG (4 μg/ml). Cells were washed twice, resuspended in 150 μl of binding buffer, and bound HCV-SP was analyzed by flow cytometry. Nonspecific fluorescence was measured by adding primary and secondary antibodies in the absence of HCV-SPs to cells. The mean fluorescence intensity (MFI) of bound HCV-SPs was determined after subtracting the nonspecific fluorescence value.

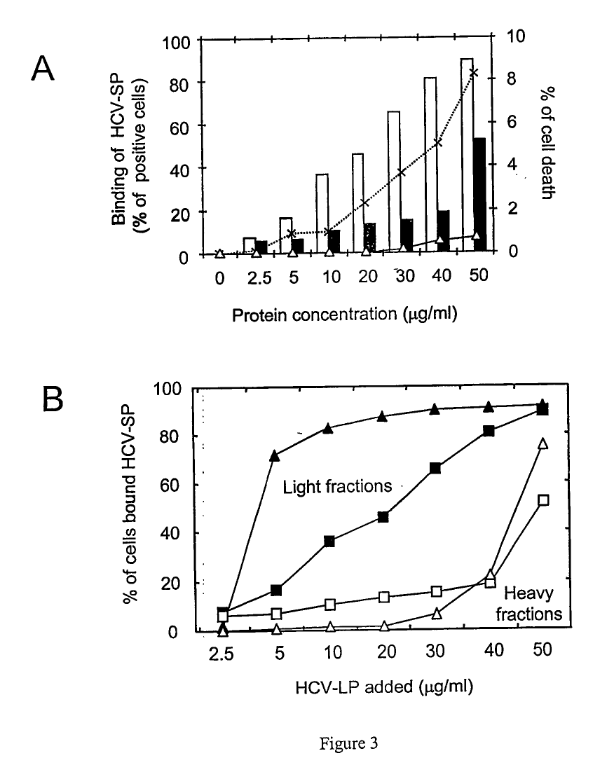

As shown in FIG. 3A, the binding of the light fraction of HCV-SP occurred in a dose-dependent manner. In contrast, very little binding was observed with the heavy fraction and only at high concentration. In addition, a slight cell toxic effect was observed with this latter fraction. This may be due to the presence of insoluble aggregates that were less recognized by conformational antibodies with ELISA and also with inmmunoblot. It is known that expression of E1 and E2 glycoproteins in mammalian cells also produced high molecular weight, disulfide-linked aggregates. The binding of HCV-SP and HCV-SP/p7⁻ preparations were compared. Binding was observed with lower concentration of the light fraction of both preparations (FIG. 3B), whereas heavy fractions of both HCV-SP and HCV-SP/p7⁻ displayed some binding activity only at the highest concentrations (50 μg/ml).

### Example 5

### Binding of HCV-SP to Primary Human Hepatocytes, HepG2, and Molt-4 Cells

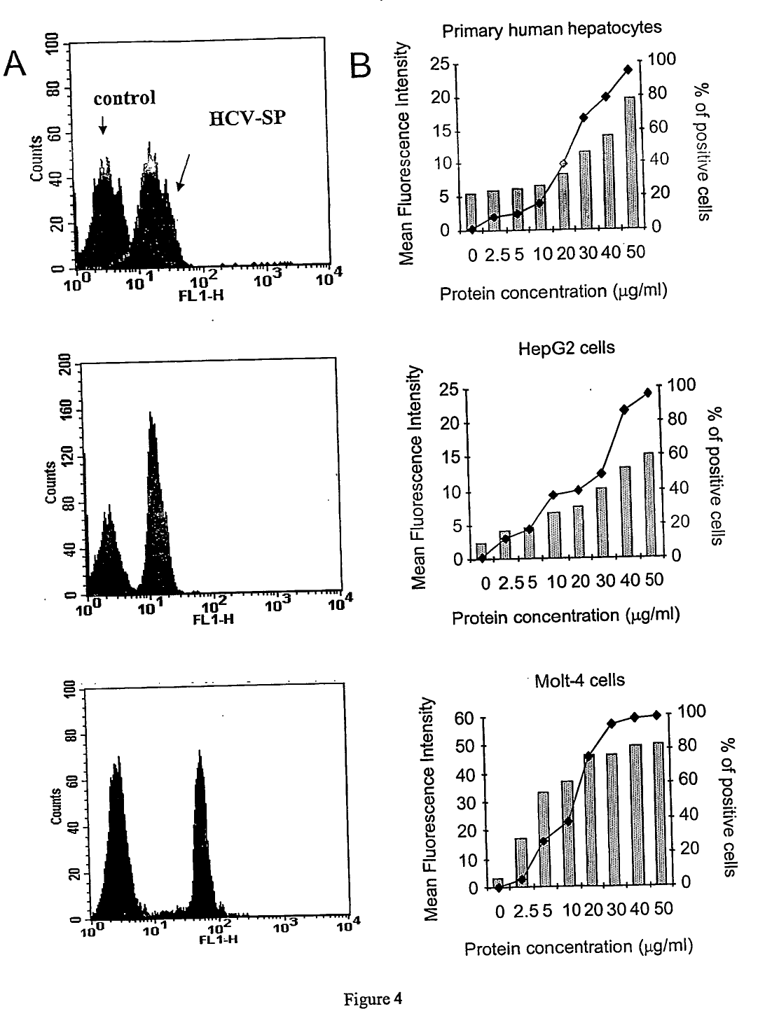

The ability of HCV-SP to bind various target cells was analyzed by flow cytometry (FIG. 4) as in Example 4. Specific HCV-SP binding was found in human hepatic cells (primary human hepatocytes and HepG2 cells) and human T cells (Molt-4 cells), but not in mouse fibroblasts (3T3-L1 cells). The binding of HCV-SP to target cells occurred in a dose-dependent manner in the various cell types (FIG. 4B). The results of analysis of the FACS data expressed in percentage of positive cells and mean fluorescence intensity correlated well (FIG. 4B).

### Example 6

### Effect of Calcium and ASGP-R Ligands on HCV-SP Binding

It was tested whether asialation of HCV envelope glycoproteins plays a role in binding of HCV-SP to hepatic cells. The asialo-glycoprotein receptor (ASGP-R) is a C-type (calcium-dependent) lectin that is most commonly found in the liver, although it is also expressed in other tissues. It has been implicated in the clearance of asialo-glycoproteins, i.e. desialated or galactose-terminal glycoproteins, from the circulation by receptor-mediated endocytosis. This receptor consists of a hetero-multimer of two homologous subunits, hH1 and hH2. Each subunit is subdivided into four functional domains: the cytosolic domain, the transmembrane domain, the stalk, and the carbohydrate recognition domain (CRD). The CRD of hH1 requires three calcium ions for proper binding conformation and sugar binding.

The cell binding assay described in Example 4 was used, but modified as described below: cells were pre-incubated with various ASGP-R ligands prior to the addition of HCV-SP. 19S-Tg fraction (Tg=thyroglobulin) contains Tg-dimers (apparent molecular weight of 660 kDa) that have a sedimentation coefficient of 19S by ultracentrifugation. Crude Tg was extracted from bovine thyroid gland and 19S-Tg was purified by column chromatography, as previously described. Orosomucoid and 19S-Tg were incubated with agarose bead-linked neuraminidase, as recommended by the manufacturer (Sigma). After centrifugation, protein concentration of the supernatants containing asialo-orosomucoid and asialo-Tg was determined. All pre-incubation steps were performed for 2 hr at 4° C.

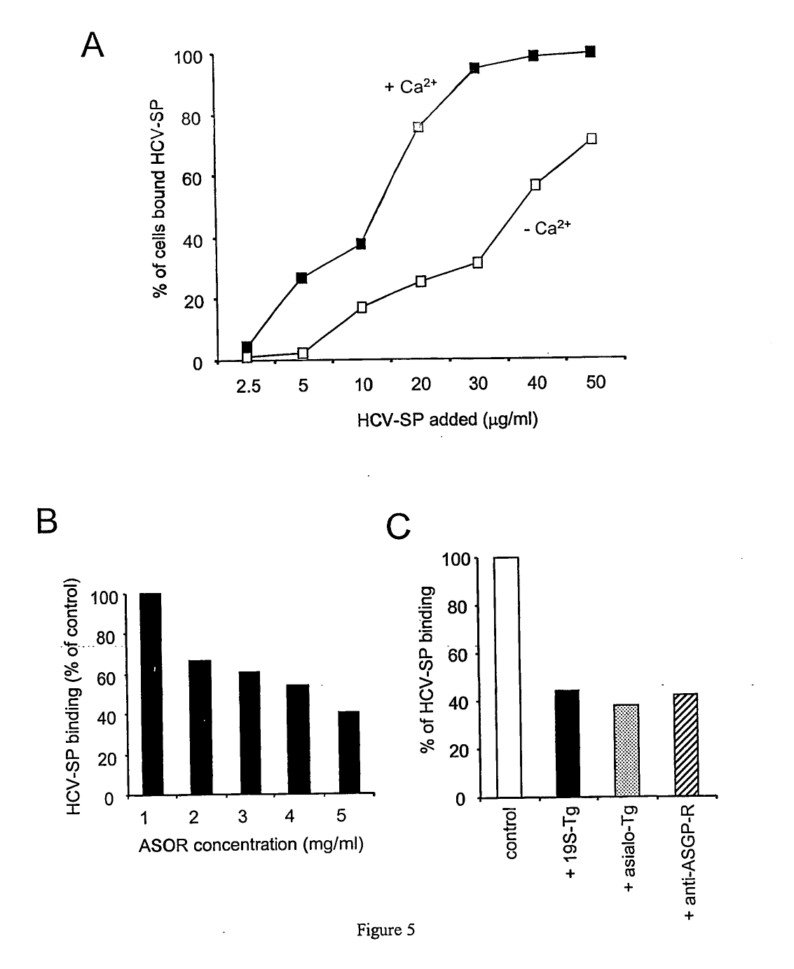

Since ASGP-R binding is calcium-sensitive, it was first asked whether HCV-SP binding to cells occurred in a calcium-dependent manner. The simultaneous removal of calcium from the binding medium together with the addition of 5 mM of the calcium chelator EGTA reduced HCV-SP binding to Molt-4 and HepG2 cells (FIG. 5A), results consistent with ASGPR being involved in HCV-SP binding. To test more directly whether the ASGP-R mediates HCV-SP binding to hepatic cells, primary human hepatocytes and HepG2 cells were pre-incubated with several ASGP-R ligands. As shown in FIG. 5B, asialo-orosomucoid, a high affinity ligand of the ASGP-R in the liver, inhibited HCV-SP binding to HepG2 cells in a dose-dependent manner. Also, pre-incubation of cells with polyclonal antibody against a peptide of the CRD of hH1 subunit of the ASGP-R resulted in the decreased on HCV-SP binding to HepG2 cells (FIG. 5C). This was not observed with preimmune antibody (not shown).

Thyroglobulin (Tg) has been previously reported to bind the ASGP-R. 19S-Tg and its desialated form (asialo-Tg) both inhibited HCV-SP binding to HepG2 cells. At lower concentration, asialo-Tg (0.4 mg/ml) showed the similar or higher inhibitory effect on HCV-SP binding as of 19S-Tg (at 1 mg/ml); desialated Tg is indeed known to have a higher affinity to the ASGP-R than 19S-Tg. Inhibition of binding was not stronger than 60-70%. It is therefore possible that additional binding site of HCV-SP exists that is neither competed by ASGP-R ligands nor sensitive to calcium.

### Example 7

### Internalization of Radio-Labeled HCV-SP in HepG2 Cells

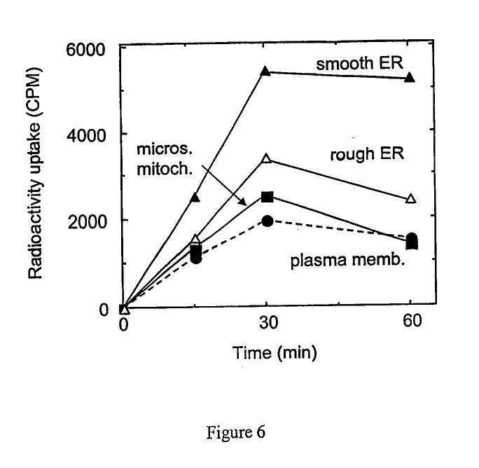

The question was then asked, after binding to cell surface receptor, HCV-SP could be internalized into human hepatic cells? To do this, Sf9 cells (5×10⁸ cells) were infected with Bac-HCV 1a.S (MOI 5) in Sf900 medium containing 0.5% FBS at 27° C. for 4 hr. Cells were pelleted, washed once with starvation medium (Sf900 medium minus cysteine and methionine), and then cells were grown in this medium for 24 hr. Then, 2 mCi of Redivue Pro-Mix [³⁵S]-methionine and cysteine mix were added to the medium and cells were further incubated for 24 hr. The labeling medium was discarded; cells were washed once with Sf900 medium and resuspended in Sf900 medium. HCV-SP (now radiolabeled) were harvested at 3 days post infection. The internalization experiment was then performed as follows: 100 μg [³⁵S]-HCV-SP was used/2×10⁸ cells/well in a 6-well plate. Cells were directly incubated at 37° C. for 15, 30, and 60 min. Cells were then harvested, disrupted and submitted to cell fractionation with sucrose gradient ultracentrifugation, resulting in four fractions corresponding to four membrane-enriched cell compartments. FIG. 6 shows that radioactivity was detected in the various compartments even after a short incubation with cells. After 15 min, the increasing amount of radioactivity was observed in all cellular compartments (plasma membrane<micros.mitoch.<SER<RER), suggesting the incorporation of labeled HCV-SP occurred in this order. After 30 min incubation, the amount of radioactivity had reached a steady state in the SER, while it started to decrease in the other intracellular compartments, suggesting that the majority of radio-labeled HCV-SP has reached the smooth endoplasmic reticulum-enriched compartment.

### Example 8

### Internalization of Dye-Labeled HCV-SP in HepG2 Cells and Co-Localization with ASGP-R GFP-hH1

It was then asked whether ASGP-R was involved in internalization. For this purpose, a clone of stable transfected HepG2 cells expressing a fusion protein between the hH1 subunit of ASGP-R and the green fluorescent protein (GFP-hH1/HepG2 cells) was established. To establish such cells, a GFP-ASGP-R construct was obtained by cloning the PCR amplimer coding for ASGP-R hH1 subunit into pcDNA3.1/NT-GFP-Topo vector (Invitrogen Corporation; Carlsbad, Calif.). Briefly, cytoplasmic RNA extracted from HepG2 cells was subjected to reverse transcription, then PCR with specific primers to obtain DNA fragments coding for hH1. The pcDNA3.1/NT-GFP-hH1 construct was verified by sequencing for correct sequence and alignment. Transient transfection experiments were performed to confirm the expression of green fluorescent protein (GFP)-hH1 fusion protein. By laser scanning confocal microscopy (LSCM) analysis, a green fluorescent signal was detected in few cells, predominating at the levels of Golgi apparatus and plasma membrane, but was also detected in other cell structures, such as vesicles (not shown). HepG2 cells were then transfected with this plasmid construct using lipofectamine-Plus and after a few days, selection antibiotic was added into the culture medium. Stable transfectants were obtained and the most positive cells were sorted using a Beckman-Coulter system.

Also used were HCV-SP that were labeled with dye. HCV-SP was labeled with 4 μM CellTracker CM-DiI (Molecular Probes; Eugene, Oreg.) in TNC buffer for 1 hr at 4° C. in the dark. Dye-labeled HCV-SP was purified through a 30% sucrose cushion at 100,000×g for 3 hr; the pellet was resuspended in TNC buffer containing 1% BSA and protease inhibitors. HepG2 cells were seeded into sterile glass chamber slides one day before the assay. Cells were incubated with labeled HCV-SPs in serum-free DMEM at 4° C. for 30 min, followed by incubation at 37° C. for 5, 15, or 30 min. Cells were rinsed once with ice-cold PBS and fixed with 4% paraformaldehyde in PEM buffer (80 mM PIPES-KOH, pH 6.8, 5 mM EGTA, 2 mM MgCl₂) for 30 min on ice. Cells were then rinsed three times with PEM buffer and slides were mounted with DAPI/antifade system and kept at dark at 4° C. until LSCM analysis was performed. Cells were analyzed with a LSCM (Leica, TCS SP) coupled with a DMIRBE inverted epifluorescent microscope. Wavelengths used to analyze GFP and CM-DiI staining were 499 and 553 nm for excitation, and 519 and 570 nm for emission, respectively.

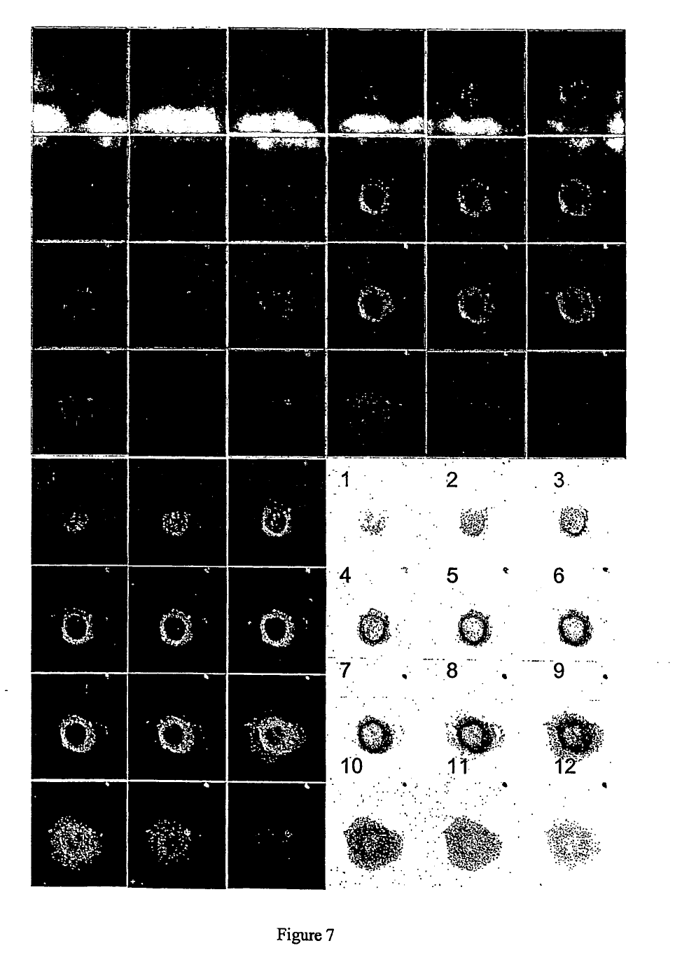

In the transfected HepG2, without added virus, some GFP signal was visible in the endoplasmic reticulum area, but mostly in the Golgi apparatus area, suggesting that GFP-hH1 subunit was properly glycosylated before targeting to the plasma membrane. Following incubation of cells with CM-DiI-labeled HCV-SP (red), co-localization was analyzed by LSCM. It was observed that, after uptake, this material accumulated in the cell area surrounding the nucleus. Moreover, by superimposing the pictures obtained in green and red channels, it was observed that there was a clear co-localization with recombinant GFP-hH1 (FIG. 7). This suggests that HCV-SP not only entered HepG2 cells, but also that it was targeted toward an area surrounding the nucleus, simultaneously with the hH1 subunit of ASGP-R.

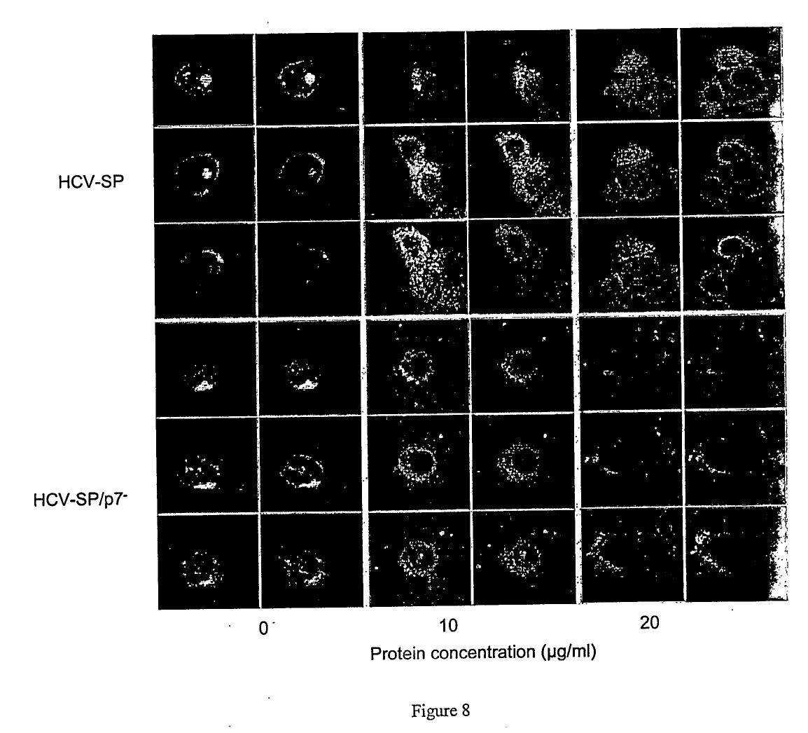

Furthermore, as shown in FIG. 8, the incubation at 37° C. of dye-labeled HCV-SP with GFP-hH1/HepG2 cells was followed by a dose-dependent uptake of the labeled material. The intensity of HCV-SP/p7⁻ uptake was less than that observed with HCV-SP preparation (FIG. 8). Finally, no uptake of dye-labeled HCV-SP was observed in a cell line of human thyrocytes (Aro cells) that do not express ASGP-R (data not shown). In addition, using a dye-labeled control preparation obtained by expressing recombinant β-glucuronidase with a baculovirus construct (bac-GUS), no uptake was observed in HepG-2 or HuH-7 cells (not shown), both well known to express ASGP-R at a high level.

### Example 9

### Binding of HCV-SP to Transfected 3T3-L1 Cells Expressing the Human Liver ASGP-R Subunits

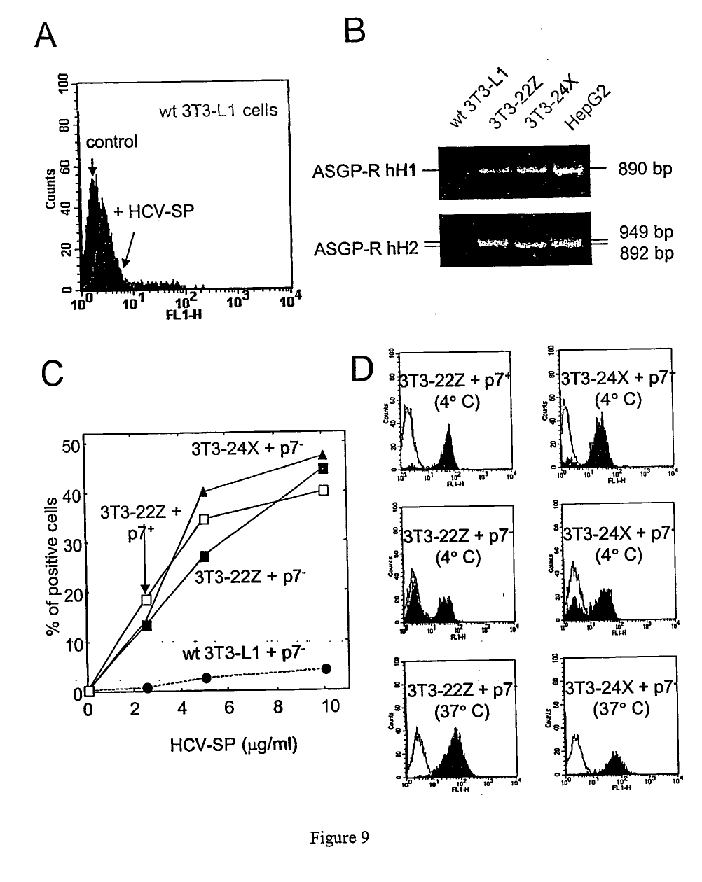

3T3-L1 cells, a cell line of mouse fibroblasts that do not bind HCV-SPs (FIG. 9A) was chosen to express the human hepatic ASGP-R (subunit hH1 and hH2). Stable ASGP-R-transfected cells (3T3-22Z) were obtained (FIG. 9B) as follows: 3T3-L1 cells were co-transfected with plasmid constructs coding for two full-length subunits of the human hepatic ASGP-R (hH1 and hH2) that have previously been shown to both be targeted to the plasma membrane in HepG2 cells. Briefly, cytoplasmic RNA extracted from HepG2 cells was subjected to reverse transcription, then PCR with specific primers to obtain cDNA fragments coding for hH1 and hH2. To allow simultaneous selection of stable transfected cells expressing both subunits, two mammalian expression vectors (pcDNA3.1-Zeo and -Neo; Invitrogen) were used. Each hH1 or hH2 cDNA fragment was inserted into one distinct vector allowing its expression under the control of a CMV promoter. The correct sequences of both constructs were verified by sequencing. 3T3-L1 cells were then transfected with both constructs simultaneously using Lipofectamine-Plus according to protocol provided by the manufacturer (Gibco-BRL/Life Technologies, Gaithersburg, Md.). Three days post-transfection, cells were passed and grown under G-418 and Zeocin selection. Upon several passages, stable 3T3-L1 transfectants were obtained. Total RNA was extracted from those cells and cDNA was synthesized by reverse transcription; PCR experiments were then performed using the same pairs of primers as above. One amplimer was detected for each PCR (hH1 or hH2) in those cells (3T3-22Z); agarose gel analysis showed that each amplimer had the same size as the corresponding amplimer obtained in HepG2 cells, whereas no amplimer was detected in 3T3-L1 parental cells. In addition, a variant was obtained, of fall length ASGP-R hH2 subunit lacking part of hH2 cytoplasmic domain (non-functional variant) but is still targeted to plasma membrane in HepG2 cells. Another stable-transfected cell line co-expressing hH1 and the hH2 variant was then established (3T3-24X).

The cells were then tested for HCV-SP binding. As shown in FIG. 9C, both HCV-SP preparations (added at low concentration: 2.5-10 μg/ml onto 10⁴ cells) bound to the ASGP-R expressing cells in a dose-dependent manner (13.23-44.46% of positive cells). Another clone of ASGP-R-transfected cells (3T3-24X) was established, expressing both hH1 and a variant of hH2 (FIG. 9B) that lacks part of its cytoplasmic domain (hH2′); the absence of this domain impairs cell trafficking of hH2′ subunit, but does not affect the binding domain. HCV-SP/p7⁻ preparation also bound to 3T3-24X cells (FIG. 9C).

### Example 10

### Internalization of HCV-SP into Transfected 3T3-L1 Cells Expressing the Human Liver ASGP-R

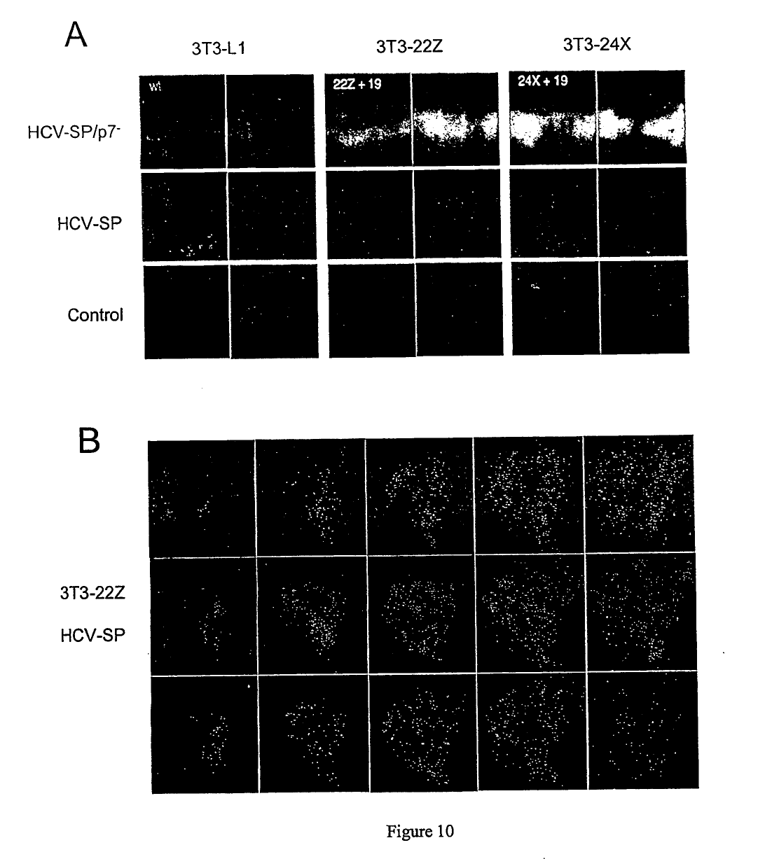

Parental 3T3-L1 cells, and cell clones 3T3-22Z and 3T3-24X were used to study whether the expression of ASGP-R, not only allowed non-permissive cells to bind HCV-SP, but also rendered them permissive for HCV-SP internalization. FIG. 10A shows that parental 3T3-L1 cells (wild type) did not uptake dye-labeled HCV-SP or HCV-SP/p7⁻. Interestingly, as 3T3-22Z cells do bind both HCV-SP and HCV-SP/p7⁻, only dye-labeled HCV-SP uptake was observed (FIG. 10B), but not dye-labeled HCV-SP/p7⁻ uptake (FIG. 10A). This correlates with the lesser uptake of HCV-SP/p7⁻ observed in HepG2 cells, in comparison to HCV-SP. Finally, 3T3-24X cells that also bind both HCV-SP and HCV-SP/p7⁻, did not uptake any of the two dye-labeled HCV-SPs FIG. 10A).

### Example 11

### Second Method of Purification—Heterogeneous HCV-Like Particles (HCV-LP)

Sf9 cells, grown at 27° C. in Sf900 medium (Gibco-BRL/Life Technologies, Gaithersburg, Md.) were infected with recombinant baculovirus at a multiplicity of infection (MOI) of 5-10, and cells were harvested at day 3 post-infection. All purification steps were carried out on ice. Cells were washed once with ice-cold 10 mM Tris-HCl pH 7.4, 150 mM NaCl, 1 mM CaCl₂ (TNC) buffer containing 1 mM Pefabloc SC and a cocktail of EDTA-free protease inhibitors (Roche, Indianapolis, Ind.), and resuspended in TNC buffer containing 0.25% digitonin and protease inhibitors. Cells were homogenized and let sit on ice with gentle agitation and monitored for cell lysis by trypan blue exclusion. Cell lysate was centrifuged to remove nuclei debris and plasma membrane, and the supernatant was pelleted over 30% sucrose cushion. The pellet was resuspended in TNC buffer, and applied onto a 10.5 ml of 20-60% sucrose gradient in SW41 tubes (Beckman) and centrifuged at 100,000×g for 16 hours. One-milliliter fractions were collected from the top of the tube and tested for E1, E2 and core proteins by ELISA and Western blot. Fractions containing HCV-LPs were stored at −70° C. Protein concentration was determined using Coomassie Plus protein assay reagent (Pierce, Rockford, Ill.) with BSA as the protein standard. The ultrastructural morphology of HCV-LPs was analyzed by cryoelectron microscopy.

### Example 12

### Characterization of HCV-LPs

The fractions collected from the sucrose gradients, as described in Example 11, were analyzed for the presence of E1, E2 and core proteins by both ELISA and Western blot, as described in Example 3.

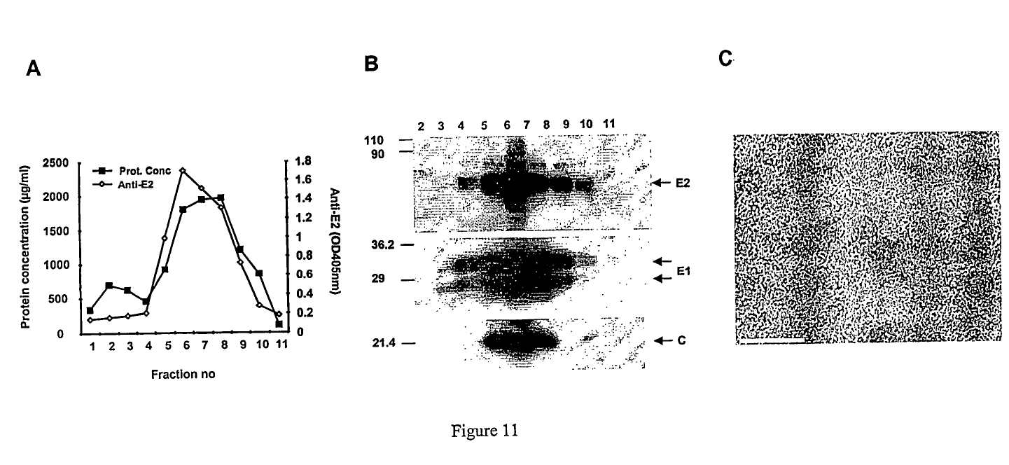

ELISA results (FIG. 11A) showed the peak of E2 reactivity was detected in fractions 6 to 8, which correspond to buoyant densities of 1.17-1.22 g/ml. Western blot analysis revealed that these fractions contain E2 protein band at ˜70 kDa, three major bands of E1 (˜33, 32 and ˜28 kDa), and a core protein band at ˜21 kDa (FIG. 11B). The presence of three bands of E1 protein reflects the different extent of N-linked glycosylation. As analyzed by cryoelectron microscopy, HCV-LPs are varying in sizes (35-49 nm in diameter) (FIG. 1C). This size difference is, in part, may be due to the difference in the amount of E1/E2 proteins incorporated into each type of particle (data not shown).

### Example 13

### Binding of HCV-LPs to Human Hepatic and Lymphoid Cell Lines

Using HCV-LPs, as isolated in Example 11, a cell-based binding assay in two formats has been developed. Both binding assays were performed at 4° C. in 100 μl of TNC buffer containing 1% BSA. For the first, indirect binding method, anti-E2 mAb was used to detect HCV-LP binding to cells. In this method, cells were incubated with various amounts of HCV-LPs for 2 h, washed twice, and cells were incubated with anti-E2 mAb (AP33) (15 μg/ml) followed by FITC goat anti-mouse IgG (4 μg/ml). Cell-bound HCV-LPs was analyzed by flow cytometry. Nonspecific fluorescence was measured by adding primary and secondary antibodies in the absence of HCV-LP to cells. The mean fluorescence intensity (MFI) of bound HCV-LP was determined after subtracting the nonspecific fluorescence value.

In the second method, the HCV-LPs were labeled with a lipophilic (CM-DiI) or nucleic acid dye (SYTO 12) and used for direct binding assay. To label, HCV-LPs were incubated with 5 μM of SYTO-12 or 1-5 μM of CM-DiI in TNC buffer at 4° C. for 15 min and re-purified through a 30% sucrose cushion to remove free dye. Cells were incubated with increasing concentrations of labeled HCV-LPs for 1 h at 4° C., washed twice, and bound (B) HCV-LPs was analyzed directly by flow cytometry. As a control for the direct binding assay, fraction prepared identically from control Bac-GUS-infected cells was labeled with the dye and used for binding assay. The MFI values of total binding (T) were based on the MFI of 100 μg/ml HCV-LPs in the absence of cells. Scatchard plot was analyzed as described.

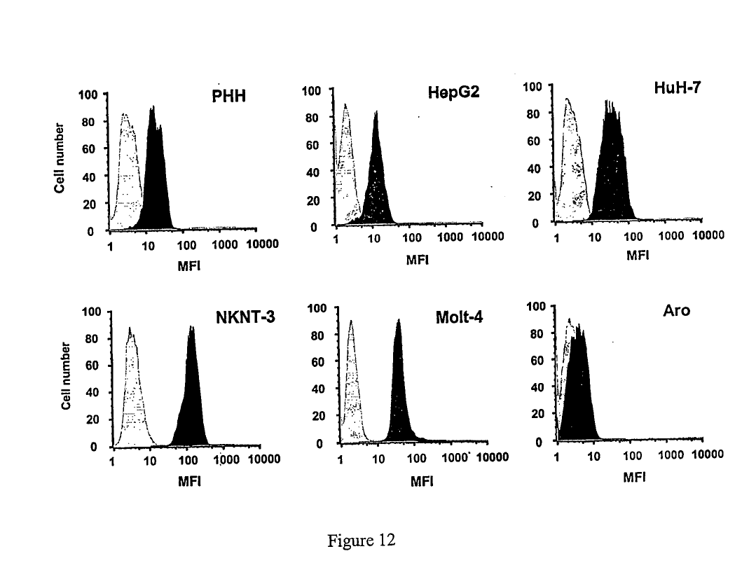

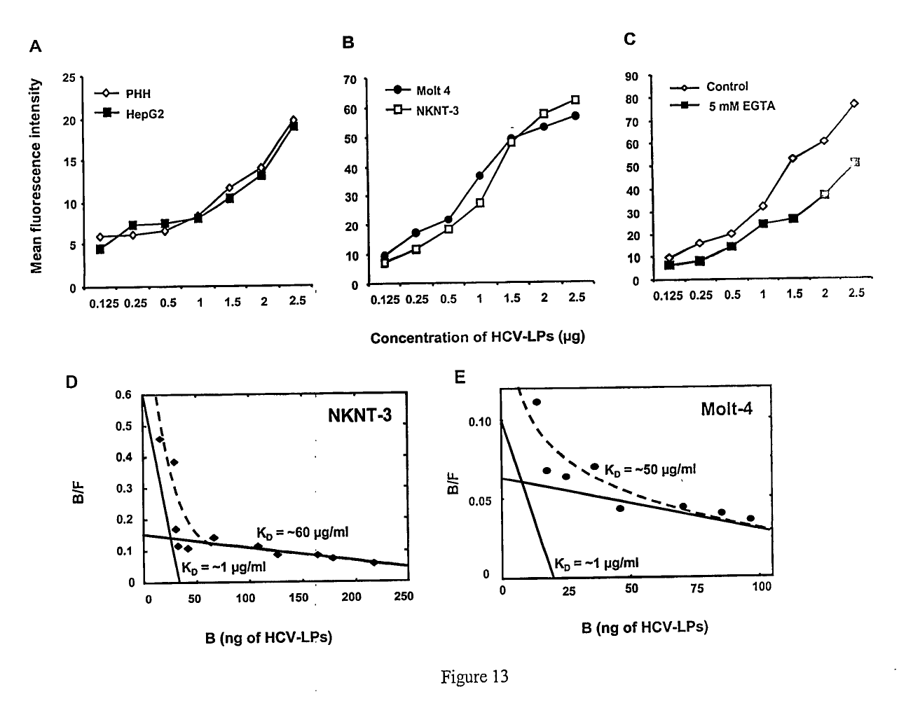

The ability of HCV-LPs to bind various target cells was analyzed by flow cytometry first using the indirect method. As shown in FIG. 12, HCV-LPs bound to hepatic (PHH, HepG2, HuH7, and NKNT-3) and T cell (Molt-4) lines, but not to thyroid cells (Aro). HCV-LPs also bound to human B cell line (Daudi), but not to Hela cells, mouse fibroblast (3T3-L1) and mouse mastocytoma cell line P815 (data not shown). Binding of HCV-LPs to target cells occurred in a dose-dependent manner and saturable (FIGS. 13A and B). HCV-LPs bound to Molt-4 and NKNT-3 cells with higher affinity than that to PHH and HepG2 cells.

Pretreatment of cells with 0.25% trypsin abolished HCV-LPs binding (data not shown), suggesting that binding of HCV-LPs to cells is mediated by cellular surface protein(s). HCV-LPs binding to cells occurred, at least partially, in a calcium-dependent manner as addition of 5 mM EGTA reduced this binding (FIG. 13C).

To estimate the affinity of HCV-LP binding to hepatic and lymphoid cells, Scatchard plot analysis was performed. Using the direct binding assay with SYTO 12-labeled HCV-LPs, it was demonstrated the presence of a biphasic binding with high and low affinities to NKNT-3 and Molt-4 cells. The high affinity binding site has a dissociation constants (K_{d}) of ˜1 μg/ml, while the lower affinity binding site has a K_{d} of ˜50-60 μg/ml (FIGS. 13D and E).

### Example 14

### Inhibition of HCV-LPs Binding by Anti-E1 and Anti-E2 mAbs

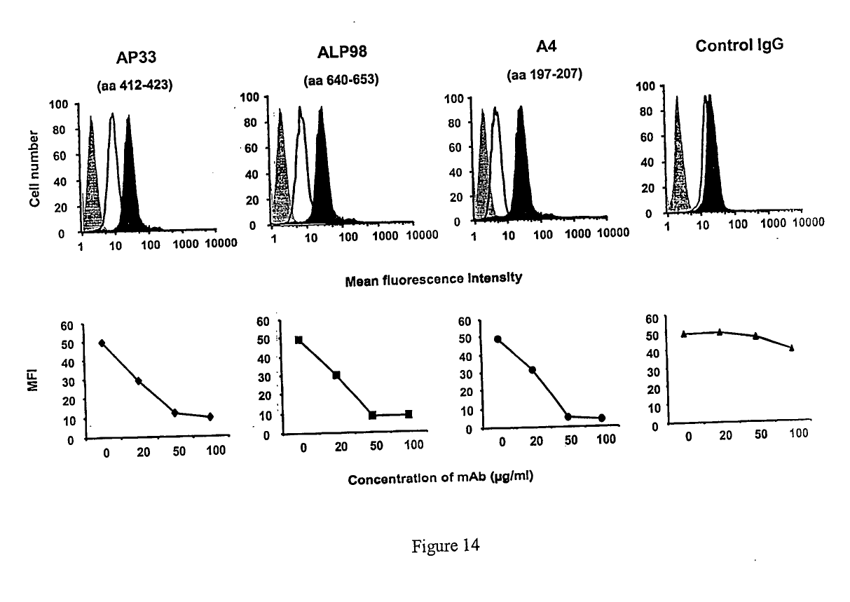

To test whether binding of HCV-LPs to cells is mediated through the envelope proteins, E1 and E2, the following study was done. SYTO 12-labeled HCV-LPs were pre-incubated with increasing amounts of anti-E2 (AP33, ALP98), anti-E1 (A4), or isotype (control) IgG for 2 h at 4° C. The HCV-LPs-antibody mixtures were then incubated with cells for 1 h. After washing, cell-bound HCV-LPs were analyzed. The results (FIG. 14) show that pre-incubation of SYTO 12-labeled HCV-LPs with anti-E2 (AP33 or ALP98) or anti-E1 (A4) mAbs inhibited HCV-LP binding to cells in a dose-dependent manner. On the other hand, neither isotype control IgG nor anti-core (data not shown) had any effect.

### Example 15

### Effect of CD81 on HCV-LP Binding

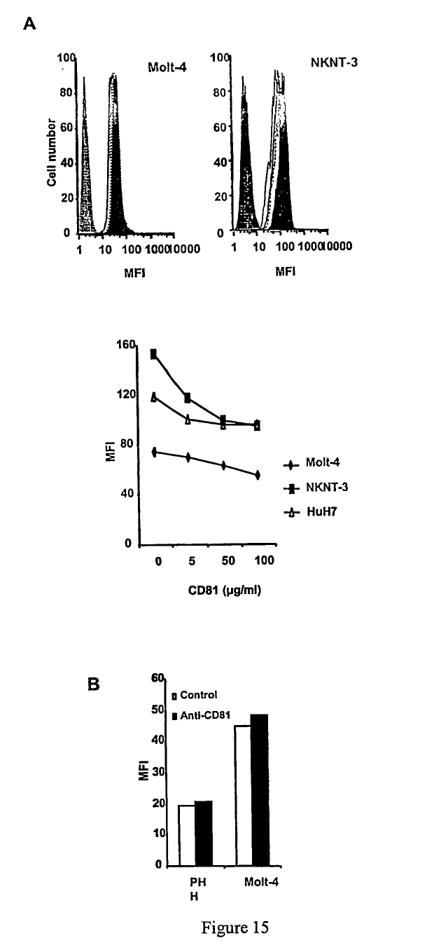

While HepG2, HuH7, NKNT-3 and Molt-4 cells all bound to HCV-LPs, significant differences in their CD81 expression existed. As assessed by RT-PCR, the strain of HepG2 cells used lacks CD81 expression, while others express CD81 (data not shown). Hence, HCV-LPs bound to HepG2 cells in a CD81-independent manner. Recombinant CD81 failed to inhibit HCV-LP binding to HuH7 cells, although it partially inhibited HCV-LPs binding to Molt-4 and NKNT-3 cells (FIG. 15A). Furthermore, anti-human CD81 mAb that had been shown to block truncated E2 binding to cells did not have any significant effect on HCV-LP binding to HuH7 and Molt-4 cells (FIG. 15B).

### Example 16

### Effect of VLDL, LDL, and HDL on HCV-LP Binding

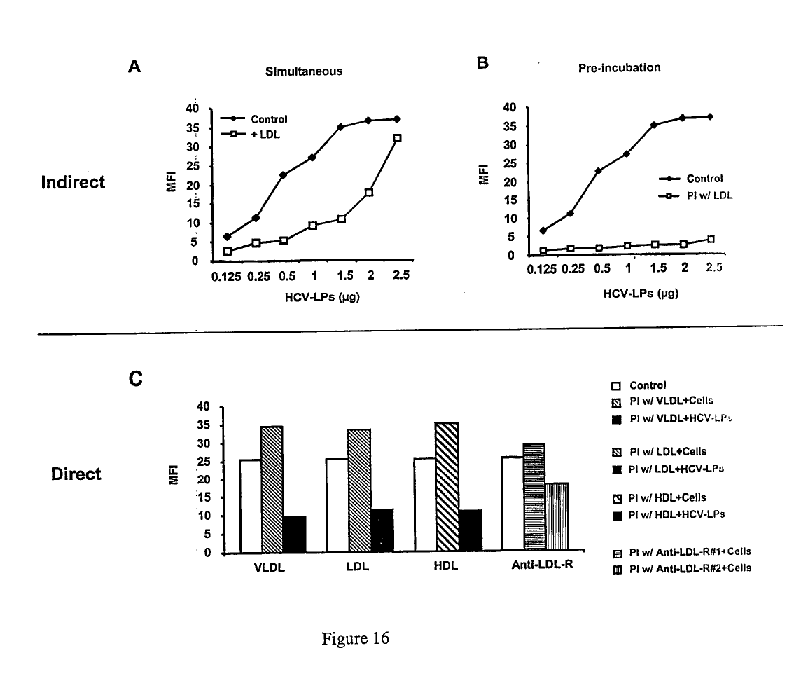

Molt-4 cells which express LDL receptors and have been used previously to characterize HCV-cell interaction were used in this study. HCV-LPs were pre-incubated with the lipoproteins before being added to cells in the indirect binding assay. It was found that LDL inhibited HCV-LPs binding when added simultaneously to cells (FIG. 16A), while pre-incubation of HCV-LPs with LDL completely abolished their binding to cells (FIG. 16B).

Previous study has proposed that association of HCV virions and β-lipoproteins in the plasma may mask the virions from circulating antibodies, and at the same time, represent one mechanism of HCV entry into cells, i.e. through the LDL receptor. There are two explanations for this finding. LDL may bind to the HCV-LPs and inhibit their binding to cells; alternatively, LDL binding to HCV-LPs may hinder the accessibility of HCV-LPs to anti-E2 mAb used in this indirect binding method. To distinguish between these two possibilities, the direct binding method was used. Cells were incubated with SYTO 12-labeled HCV-LPs. As shown in FIG. 16A, pre-incubation of labeled-HCV-LPs with LDL reduced their binding to Molt-4 cells by >50%. A similar phenomenon was observed when HCV-LPs was pre-incubated with VLDL or HDL. However, when cells were pre-incubated either with VLDL, LDL or HDL before the addition of HCV-LPs, HCV-LPs binding was slightly increased (FIG. 16C). Altogether, these results indicate that pre-incubation of HCV-LPs with VLDL, LDL and HDL resulted in lipoprotein-HCV-LPs complex that inhibited HCV-LP binding to cell. Second, the increased HCV-LP binding after pre-incubation of cells with these lipoproteins implied that HCV-LPs can also interact with cell-bound VLDL, LDL, or HDL, in addition to other cell surface molecule(s). This was confirmed by the inability of two anti-LDL-R antibodies to significantly block HCV-LP binding (FIG. 16C).

### Example 17

### Internalization of Labeled-HCV-LPs by Hepatic Cells

It was examined whether binding of HCV-LPs to cells can be followed by entry. HuH7 and NKNT-3 cells were incubated with CM-DiI or SYTO-labeled HCV-LPs at 4° C. for 30 min, followed by incubation at 37° C. for various time points. The specificity of internalization process was determined by pre-incubating dye-labeled HCV-LPs with anti-E1 and anti-E2 antibodies before added to cells. As a negative control, cells were incubated with CM DiI- or SYTO-labeled preparation from cells infected with Bac-GUS. Alternatively, Aro cells were incubated with dye-labeled HCV-LPs. Cells were fixed with 4% paraformaldehyde, washed and mounted with DAPI/antifade system. Cells were imaged on a Leica TCS SP laser-scanning confocal microscope mounted on a DMIRBE inverted epifluorescent microscope. SYTO and CM-DiI fluorescent dyes were excited by a 499 nm and 553 nm, respectively, laser lines from a water-cooled argon laser (Coherent Laser, CA). SYTO and CM-DiI fluorescence emissions were monitored at 519 and 570 nm, respectively.

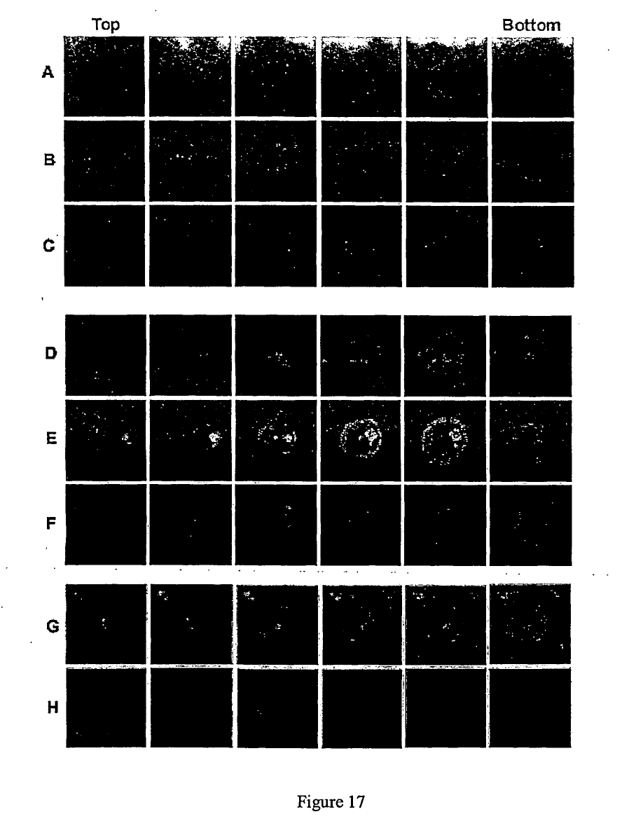

FIG. 17 showed the internalization of CM-DiI-labeled HCV-LPs by HuH7 cells as analyzed by laser-scanning confocal microscopy. This internalization was temperature-dependent as only a weak signal was detected at 4° C. (FIG. 17A), while following incubation at 37° C., a higher intensity of dye-labeled HCV-LPs was observed in the cytoplasm surrounding the nucleus (FIG. 17B). In contrast, HuH7 cells did not uptake CM-DiI-labeled Bac-GUS preparation after incubation at 37° C. (FIG. 17C). Aro cells that did not bind HCV-LPs were used as a negative control to assess the specificity of the internalization of HCV-LPs. The results showed that, Aro cells did not uptake labeled HCV-LPs (data not shown).

The ability of NKNT-3 Cells to internalize SYTO lableled-HCV-LPs was shown in FIGS. 17D to H. Following incubation at 4° C., a weak signal of SYTO-labeled HCV-LPs was found mostly surrounding the cell surface (FIG. 17D). The incorporation of dye into the cytoplasm increased when cells were incubated at 37° C. for 30 min (FIG. 17E). It was also observed that SYTO dye was found in the nucleoli, which is presumably due to the staining of the RNA-containing nucleoli by the dye released from HCV-LPs after entry. NKNT-3 cells reacted poorly with SYTO-labeled Bac-GUS preparation (FIG. 17F). To assess whether specific antibodies could inhibit HCV-LPs entry into cells, labeled HCV-LPs were pre-incubated with anti-E1/-E2 antibodies for 2 h at 4° C. HCV-LPs (in the absence of antibodies) and after pre-incubation with antibodies were then incubated with cells for 15 min at 37° C. While the control HCV-LPs were internalized by cells (FIG. 17G), pre-incubation with antibodies significantly reduced the incorporation of labeled HCV-LPs (FIG. 17H). These data suggest that E1 and E2 protein mediate HCV-LPs binding and subsequently, their entry into cells.

### Example 18

### Third Method of Purification—Homogeneous HCV-Like Particles (HCV-LP)

Sf9 insect cells were grown in Sf900 II medium containing antibiotics-antimycotics at 27° C. (125 rpm) in sterile Erlenmeyer flasks with a volume ratio <1/3. To amplify HCV recombinant baculovirus stock, insect cells were infected at an MOI 0.1 (Virus titer was determined by BAC-Pak Rapid Titer kit) and harvested at 3 days post-infection. Supernatant containing baculovirus was concentrated by centrifugation at 48,000×g for 2 h at 4° C. (SW28 rotor; Beckman). The virus pellet was resuspended in Sf900 medium and stored in small aliquots at −70° C.

The infection protocol for small-scale preparation was as follows: Sf9 cells were infected with recombinant baculovirus at an MOI of 1 or 10/cell. To ensure that cells were infected simultaneously, cells were resuspended in a small volume of medium containing the inoculum (˜10⁸ cells/5 ml) for 1 h in 125 ml sterile Erlenmeyer flask. After 1 h, without removing the inoculum, fresh Sf900 II medium (containing 0.5% fetal bovine serum and antibiotics-antimycotics solution) was added to reach a density of 2.5-5×10⁶ cells/ml. Cells were grown at 27° C. (125 rpm) and harvested after 2, 3 or 4 days incubation.

The following steps in the cell lysis protocol were performed either on ice or at 4° C.: Sf9 cells were centrifuged into a pellet by rapid centrifugation (3,500 rpm for 1-2 min, without brake) and culture medium was removed. The volume of the pellet was measured and the term “volume” in the following steps refers to pellet volume. Cells were rinsed by suspending them once in 20 volumes of ice-cold PBS×1, and then pelleted by rapid centrifugation (cf. above) and supernatant was removed. The cells were resuspended by brief vortexing or gentle pipetting in 10 volumes of ice-cold glycerol buffer (50 mM Hepes-NaOH, pH 7.4, containing 5% glycerol, 2 mM EGTA and 2 mM EDTA) and incubated on ice for 30 min; gently swirling the solution by inverting the tube once or twice every 5 to 10 min.

Cells were centrifuged at high speed to pellet the cells and if to remove the excess glycerol. The supernatants were removed and the tube walls were carefully rinsed with 2 volumes of ice-cold hypotonic buffer (10 mM Hepes-NaOH, pH 7.4, containing 1× protease inhibitor cocktail, 2 mM EGTA and 2 mM EDTA) without resuspending the pellet. Then the liquid used to rinse the tubes was removed (if necessary centrifugation was briefly done again). Cells were resuspended (no vortex, no pipetting) in 2 to 6 volumes (depending on percentage of glycerol used above) of ice-cold lysis buffer (hypotonic buffer containing 0.25% digitonin) and incubated on ice for 15 min; gently swirling the solution every 5 min. The cell lysate was centrifuged at 1,500×g for 5 min to remove cell nuclei and debris. The lysate from this step was centrifuged at 30,000×g (15,000 rpm, SW28, Beckman) for 30 min to remove membranes. The lysate from this step was then centrifuged at 100,000×g for 3 h (28,000 rpm, SW28) through 10 ml of 30% sucrose cushion to pellet VLP; [rate zonal gradient: make continuous sucrose gradient: 0.75 ml of each 20, 30, 40, 50, 60 and 66% sucrose and incubate at 37° C. for 1 h, then cool on ice]. The pellet was gently resuspended in TNC buffer (50 mM Tris-HCl, pH 7.4, containing 150 mM NaCl and 1 mM CaCl₂) plus protease inhibitor cocktail with potter (0.5 ml glass/teflon homogenizer [1 ml for maxipreps]) without foaming.

The resuspended pellet was then subjected to equilibrium centrifugation as follows: less than 0.3 ml of sample was loaded on the top of a 20-60% sucrose gradient: 0.75 ml of each 20, 30, 40 and 50% sucrose, and 1.5 ml of 60% sucrose (for 5 ml tubes of SW55, Beckman). Centrifugation was at 100,000×g (slow acceleration, without brake) for 18 h. One-half ml fractions were collected from the top of the gradient. Bands are visible from fraction 5 to 7. Protein concentration was determined using Coomassie Plus protein assay reagent with BSA as the protein standard. FIG. 2 shows profile of total protein concentration on each fraction following 20-60% sucrose gradient centrifugation. Samples were prepared from cells infected at MOI 1 and 10 and harvested at 2, 3 and 4 days post-infection as indicated. FIG. 3 shows SDS-PAGE and Western Blot analysis of gradient fractions 3-9 of HCV-LP from harvest of cells infected at an MOI of 10 and harvested 6 days post-infection. The Western Blots were probed with monoclonal antibodies specific for E2 (ALP98), E1 (A4) and the core (C1).

Alternatively, equilibrium centrifugation was performed by centrifuging on the top of a preformed sucrose gradient (cf. above) at 100,000×g for 2 h 30 min (slow acceleration without brake) or using a SW41 rotor (Beckman), the 20-60% sucrose gradient is as follows: 1.5 ml of each 20, 30, 40 and 50% sucrose, and 2.5 ml of 60% sucrose (10.5 ml tubes). Less than 0.5 ml sample was loaded and centrifuged at 100,000×g (slow acceleration, without brake) for 18 h. Collect 1 ml fractions from the top.

The virus was then collected from the collected gradient fractions by centrifuging the fractions at 100,000×g (33,000 rpm, SW55 with brake) through 1.5 ml of 30% sucrose cushion to pellet purified VLP for 90 min at 4° C.

### Example 19

### Characterization of Purified Particles

Several aspects of the HCV-LP obtained with this method were analyzed: yield of HCV-LP containing fractions (total protein concentration/ml culture), biophysical properties, immunoreactivity of HCV-LP (Western Blot) and its ultrastructure (by cryoelectron microscopy analysis).

Yield: With 30 ml culture (10⁸ cells), a maximum protein concentration of 1.2 mg/ml was obtained in the fractions with a total of≈2.2 mg protein containing core, E1 and E2 proteins.

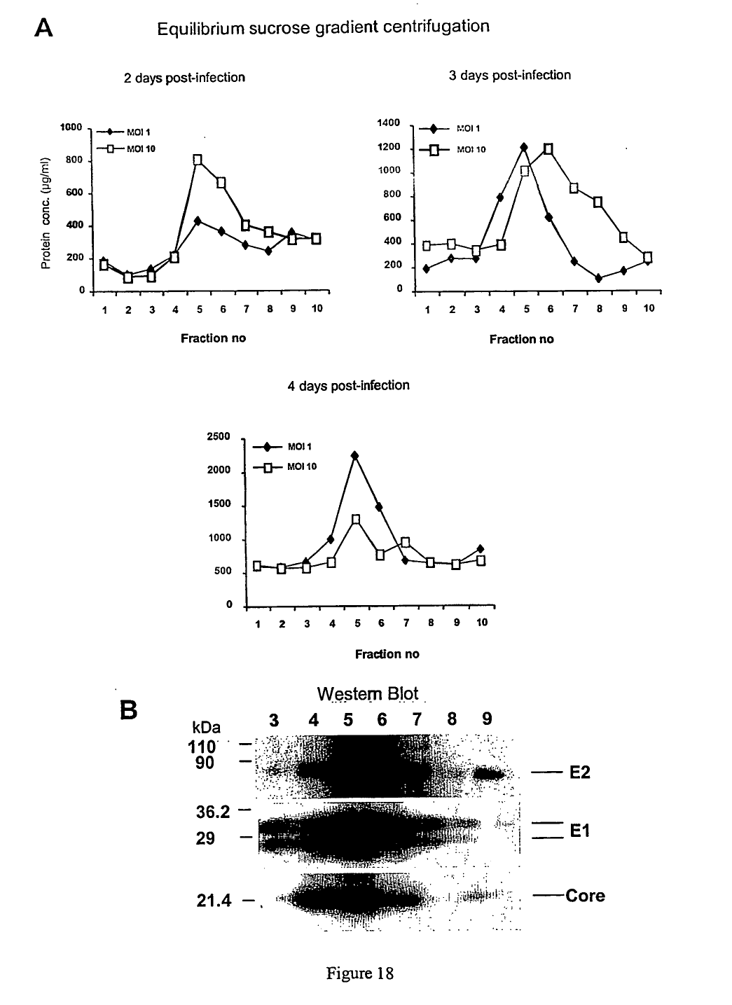

Biophysical properties: Following sucrose gradient centrifugation, HCV-LP was found at buoyant densities of 1.15-1.18 g/ml (FIG. 18A).

Immunoreactivity: The fractions collected after sucrose gradient ultracentrifugation were analyzed by Western Blot using specific anti-core, anti-E1, and anti-E2 monoclonal antibodies. The result showed that fractions 5-7 exhibited very strong reactivity to all anti-structural protein antibodies tested (FIG. 18B).

Cryoelectron microscopy: The HCV-LP preparation was so examined and homogenous double-shelled particles of ˜50 nm in diameter were observed. In addition, this preparation was ‘clean’ from impurities.

### Example 20

### Binding of HCV-LP to Target Cells

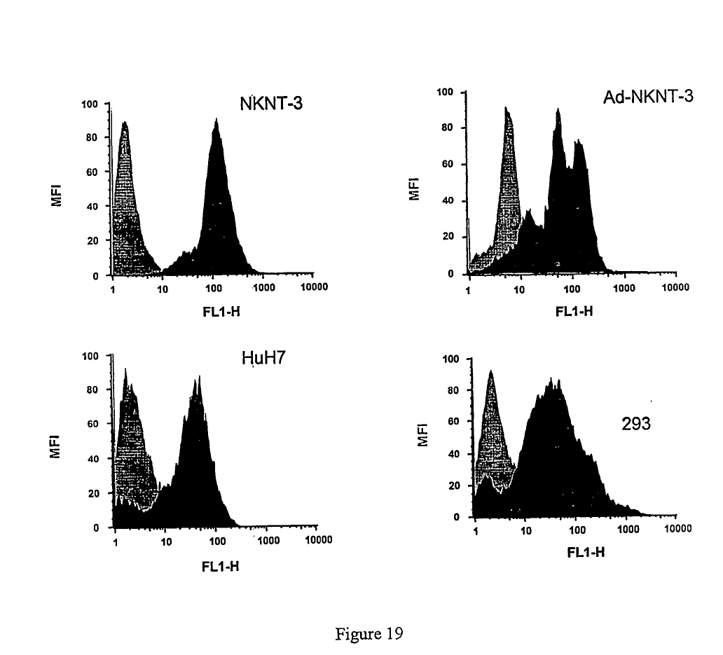

HCV-LP have been tested for its ability to bind to target cells. Human hepatic cells (HuH7) and kidney cells (293) were obtained from American Type Culture Collection. An immortalized human hepatocyte cell line (NKNT-3) and a replication-deficient recombinant adenovirus (Ad) that express the Cre recombinase tagged with a nuclear localization signal (AdCANCre) was a gift from I. J. Fox (Omaha, Nebr.). Differentiation of NKNT-3 cells to mimic normal primary hepatocytes was achieved by transduction with AdCANCre followed by selection with G418 (Ad-NKNT-3) with a slight modification. Cells were grown in Chee's Modified MEM containing 5% fetal bovine serum and were analyzed for HCV-LP binding at 3 days post-transduction. HCV-LP was directly labeled with SYTO-12 (nucleic acid dye) according to the manufacturer's protocol. Briefly, HCV-LP were incubated with 5 μM of SYTO-12 in TNC buffer at 4° C. for 15 min and re-purified through a 30% sucrose cushion to remove free dye. 2×10⁵ cells were incubated with 2.5 μg of SYTO 12-labeled HCV-LP in 50 μl TNC buffer containing 1% BSA and a cocktail of EDTA-free protease inhibitors, for 1 hr at 4° C. Cells were washed once with PBS, detached with 0.25 mM EDTA (in PBS) for 10 min at 37° C., and resuspended in binding buffer. After washing, cell-bound HCV-LP were analyzed by flow cytometry. FIG. 19 shows the results. For each cell type, the histogram shows cells in the absence of HCV-LP (gray graph) and after incubation with HCV-LP (black graph). Tie results showed that HCV-LP bind to HuH-7, NKNT-3 and HEK-293 cells in a dose-dependent manner.

### Example 21

### Inhibition of HCV-LP Binding to Cells by Anti-E2, -E2 and -Core Antibodies

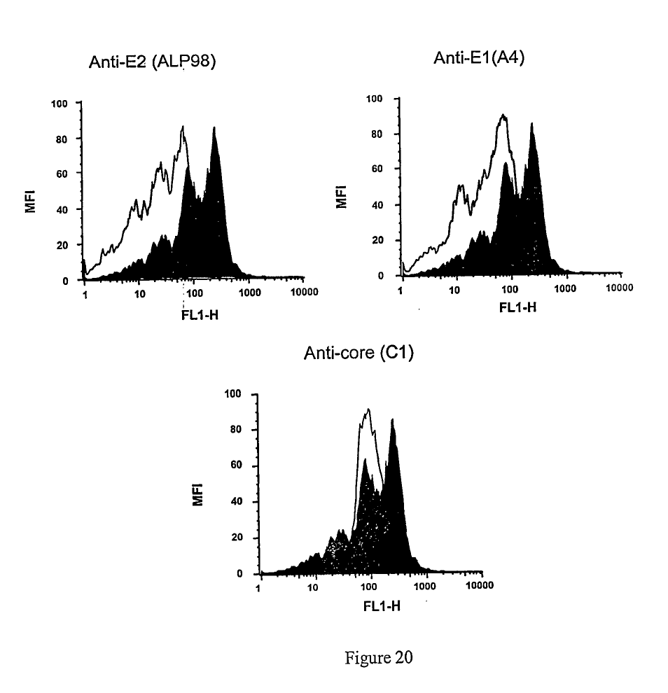

SYTO 12-labeled HCV-LP were pre-incubated with 20 μg/ml of anti-E2 (ALP98), anti-E1 (A4), or anti-C mAbs for 2 h at 4° C. and were then incubated with Ad-NKNT-3 cells for 1 h (FIG. 5, open graph). As control, cells were incubated with HCV-LP in the absence of antibodies (FIG. 20, closed graph). After washing, cell-bound HCV-LP were analyzed by flow cytometry. The inhibition of HCV-LP binding to cells by the anti-E1 and anti-E2 antibodies suggest that binding of HCV-LP to the cells is likely mediated through the envelope proteins E1 and E2. Anti-core antibodies had much less of an effect on HCV-LP binding to cells.

### Example 22

### Effect of Lipoproteins on HCV-LP Binding to Cells

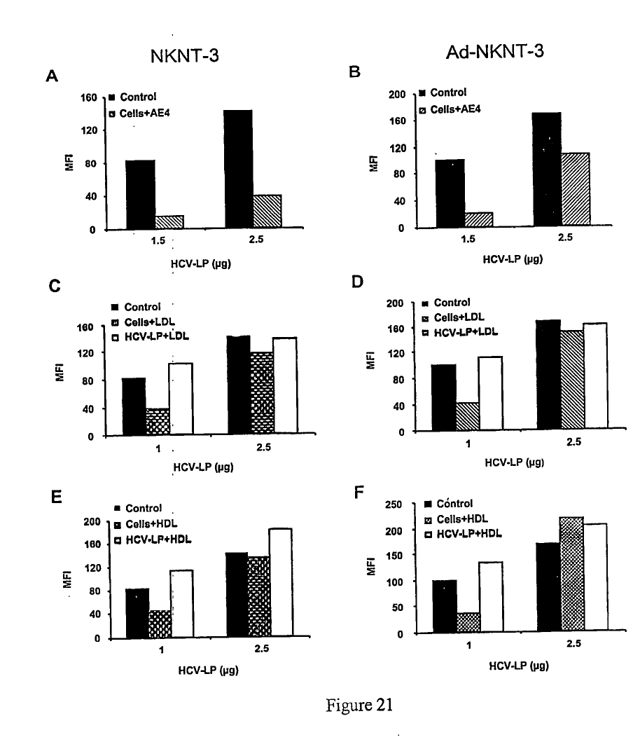

NKNT-3 cells were transduced with recombinant AdCANCre. HCV-LP binding was performed at 3 days post-transduction using 2×10⁵ cells incubated with 1.5 or 2.5 μg of SYTO 12-labeled HCV-LP (FIG. 21, closed bar) for 1 hr at 4° C., and analyzed by flow cytometry. (A, B) NKNT-3 or Ad-NKNT-3 cells were pre-incubated with apolipoprotein E4 for 2 hr at 4° C. before adding HCV-LP and incubating for another 1 hr (striped bar). (C, D) Cells were pre-incubated with 0.5 mg/ml of LDL (hatched or striped bar) or without (closed bar), as a control, before adding dye-labeled HCV-LP. Alternatively, HCV-LP were pre-incubated with LDL before adding to cells (open bar). (E, F) Cells were pre-incubated with 0.5 mg/ml of HDL before adding dye-labeled HCV-LP (hatched bar); as a control, cells were incubated with HCV-LP in the absence of LDL (closed bar). Alternatively, HCV-LP were pre-incubated with HDL before adding to cells (open bar).

### Example 23

### Effect of AGSP-R Ligands on HCV-LP Binding to Cells

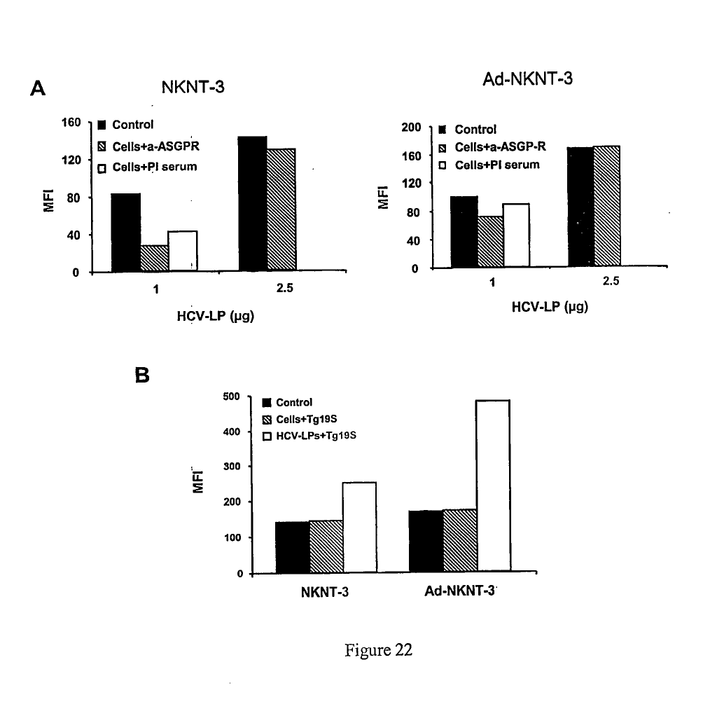

NKNT-3 cells were used as is or transduced with recombinant AdCANCre. (FIG. 22A) Cells were then pre-incubated with rabbit anti-ASGPR antibody for 2 hr at 4° C. before adding SYTO 12-labeled HCV-LP (striped bar). As control, cells were incubated with HCV-LP in the absence of anti-ASGP-R antibody (closed bar). (FIG. 22B) Cells were pre-incubated with 0.5 mg/ml of Tg 19S for 2 hr at 4° C. before SYTO 12-labeled HCV-LP was added (striped-bar). Alternatively, HCV-LP were pre-incubated with Tg 19S for 2 hr at 4° C. before added to cells (open bar).

1. A method for isolating infection defective hepatitis C virus (HCV) structural protein complexes from cells infected with a baculovirus encoding and expressing HCV structural proteins, comprising:
a) lysing the infected cells to yield a lysate;
b) adding polyethylene glycol to the lysate to form a precipitate that comprises the infection defective HCV structural protein complexes.

2. The method of claim 1 further comprising the step of fractionating the precipitate by gradient ultracentrifugation to provide a fraction comprising said complexes.

3. The method of claim 1 wherein the cells are lysed by incubating the cells in a buffer containing digitonin and protease inhibitors.

4. A preparation of infection defective HCV structural protein complexes prepared according to the method of claim 1.

5. A method for isolating infection defective hepatitis C virus (HCV)-like particles from cells infected with a baculovirus encoding and expressing HCV structural proteins, comprising:
a) lysing the-infected cells to yield a lysate;
b) centrifuging the lysate through a cushion comprising a monosaccharide, disaccharide, or polysaccharide to provide a pellet comprising a preparation of HCV-like particles, wherein said preparation contains HCV-like particles that are heterogenous in size.

6. The method of claim 5 further comprising the step of fractionating the pellet by gradient centrifugation to provide a fraction comprising said preparation of heterogenous HCV-like particles.

7. The method of claim 5 wherein the cells are lysed by incubating the cells in a buffer containing digitonin and protease inhibitors.

8. A preparation of infection defective HCV-like particles prepared according to the method of claim 5.

9. A method for isolating infection defective hepatitis C virus-like particles from cells infected with an expression system encoding and expressing HCV structural proteins, comprising:
a) incubating the cells in a hypertonic solution;
b) incubating the cells in a hypotonic solution;
c) lysing the cells to yield a lysate; and
d) centrifuging the lysate through a cushion to provide a pellet comprising a preparation of HCV-like particles that are substantially homogeneous, wherein said HCV-like particles are approximately 50 nm in diameter.

10. The method of claim 9 further comprising the step of fractionating the pellet by gradient ultracentrifugation to provide a fraction comprising said substantially homogeneous HCV-like particles.

11. The method of claim 9 wherein the cells are lysed by incubating the cells in a buffer containing digitonin and protease inhibitors.

12. The method of claim 9 wherein the HCV-like particles comprise E1 and E2-p7 proteins of HCV.

13. The method of claim 9 wherein the HCV-like particles comprise E1 and E2 without p7 proteins of HCV.

14. A preparation of infection defective HCV-like particles prepared according to the method of claim 9.

15. A method of detecting antibodies reactive with hepatitis C virus comprising in a subject:
a) incubating a sample from the subject with the HCV-like particles of claim 8 or claim 14;
b) assaying for the formation of complexes between antibodies in the sample and the hepatitis C virus-like particles, wherein formation of said complexes indicates that the sample contains antibodies that are reactive with hepatitis C virus.

16. A method of identifying a substance that inhibits binding of hepatitis C virus to its host cells comprising:
a) contacting cells capable of binding hepatitis C virus with a candidate substance;
b) incubating the cells with the HCV-like particles of claim 8 or claim 12, and
c) assaying for a reduction in binding of the HCV-like particles to the cells in the presence of the candidate substance, wherein a candidate substance that reduces binding of the HCV-like particles to the cells is capable of inhibiting binding of HCV to the host cells.

17. A method for treating a subject exhibiting symptoms of HCV infection comprising administering to the subject a substance that interferes with binding of the HCV-like particles of claim 8 or claim 14 to cells.

18. The method of claim 17 wherein the substance is an antibody that is immunoreactive with the asialoglycoprotein receptor.

19. The method of claim 17 wherein the substance is thyroglobulin.

20. A kit for detecting hepatitis C virus, antibodies reactive with hepatitis C virus, or substances that interfere with binding of hepatitis C virus to cells comprising:
a) cells transfected with one or more expression systems encoding and expressing one or more receptors to which hepatitis C virus is capable of binding; and
b) one or more preparations selected from the group consisting of the preparation of claim 4, the preparation of claim 8, the preparation of claim 14.

21. The kit of claim 20 wherein the cells are transfected with an expression system encoding an asialoglycoprotein receptor.

22. A method of inducing production of antibodies immunoreactive with HCV in an animal, comprising administering a preparation selected from the group consisting of the preparation of claim 4, the preparation of claim 8, and the preparation of claim 14, or a combination of said preparations to the animal.
